(19) United States
(12) Patent Application Publication
(10) Pub. No.: US 2008/0035765 A1
(43) Pub. Date: Feb. 14, 2008

(54) Environmental system including a micromechanical dispensing device

(75) Inventors: Joel A. Kubby, Rochester, NY (US); Nancy Y. Jia, Webster, NY (US); Edward Francis, Pittsford, NY (US); Robert M. Duffner, Penfield, NY (US); Jun Ma, Penfield, NY (US); Cathie J. Burke, Rochester, NY (US); James C. Diehl, Rochester, NY (US)

Correspondence Address: PATENT DOCUMENTATION CENTER, XEROX CORPORATION, 100 CLINTON AVE., SOUTH, XEROX SQUARE, 20TH FLOOR, ROCHESTER, NY, 14644, US

(73) Assignee: Xerox Corporation

(21) Appl. No.: 11/801,010

(22) Filed: May 8, 2007

Related U.S. Application Data
(62) Division of application No. 10/827,922, filed on Apr. 20, 2004.

(51) Int. Cl. A61L 9/14

(52) U.S. Cl. 239/690; 239/302; 239/71

(57) ABSTRACT

An environmental system includes a micromechanical dispensing device. The environmental system comprises an environmental system controller arranged to communicate with one or more included environmental air units. The one or more environmental air units are arranged to alter or control one or more physical properties of an atmosphere. The environmental system controller is further arranged to communicate with an included micromechanical dispensing device. The micromechanical dispensing device is arranged to dispense at least one fluid into the atmosphere.

### CROSS-REFERENCE TO RELATED APPLICATION

In accordance with the provisions of 35 U.S.C. section 121, this is a divisional application of its commonly-assigned parent application Ser. No. 10/827,922 filed 20 Apr. 2004 by the same inventors hereof, now pending. The disclosure of the prior parent application is totally incorporated herein by reference. This divisional application claims the priority benefit of the prior parent application under the provisions of 35 U.S.C. sections 120 and 121. This divisional application is being filed in response to a restriction requirement in the prior parent application.

### INCORPORATION BY REFERENCE OF OTHER U.S. PATENTS

The disclosures of the following twenty-six (26) U.S. patents are hereby incorporated by reference, verbatim, and with the same effect as though the same disclosures were fully and completely set forth herein:

Carole C. Barron et al., “Chemical-mechanical polishing of recessed microelectromechanical devices,” U.S. Pat. No. 5,919,548 (hereinafter “Barron '548”);

Carole C. Barron et al., “Method for integrating microelectromechanical devices with electronic circuitry,” U.S. Pat. No. 5,963,788 (hereinafter “Barron '788”);

John M. Bloemer, “Humidifier with reversible housing and distribution tray overflow,” U.S. Pat. No. 6,572,085 (hereinafter “Bloemer”);

Edward M. Carrese et al., “Ink tank with securing means and seal,” U.S. Pat. No. 6,390,615 (hereinafter “Carrese”);

Steven T. Cho, “Microfluidic valve and system therefor,” U.S. Pat. No. 6,561,224 (hereinafter “Cho”);

Charles P. Coleman et al., “Method of fabricating a fluid drop ejector,” U.S. Pat. No. 6,127,198 (hereinafter “Coleman '198”);

Charles P. Coleman et al., “Fluid drop ejector,” U.S. Pat. No. 6,318,841 B1 (hereinafter “Coleman '841”);

Anthony J. Fariono et al., “Method for photolithographic definition of recessed features on a semiconductor wafer utilizing auto-focusing alignment,” U.S. Pat. No. 5,783,340 (hereinafter “Fariono”);

Frank C. Genovese et al., “Magnetically actuated ink jet printing device,” U.S. Pat. No. 6,234,608 B1 (hereinafter “Genovese”);

Arthur M. Gooray et al., “Magnetic drive systems and methods for a micromachined fluid ejector,” U.S. Pat. No. 6,350,015 B1 (hereinafter “Gooray '015”);

Arthur M. Gooray et al., “Micromachined fluid ejector systems and methods,” U.S. Pat. No. 6,367,915 B1 (hereinafter “Gooray '915”);

Arthur M. Gooray et al., “Fluid ejection systems and methods with secondary dielectric fluid,” U.S. Pat. No. 6,406,130 B1 (hereinafter “Gooray '130”);

Arthur M. Gooray et al., “Bi-directional fluid ejection system and methods,” U.S. Pat. No. 6,409,311 B1 (hereinafter “Gooray '311”);

Arthur M. Gooray et al., “Micromachined fluid ejector systems and methods having improved response characteristics,” U.S. Pat. No. 6,416,169 B1 (hereinafter “Gooray '169”);

Arthur M. Gooray et al., “Electronic drive systems and method,” U.S. Pat. No. 6,419,335 B1 (hereinafter “Gooray '335”);

Joel A. Kubby et al., “Micro-electro-mechanical fluid ejector and method of operating same,” U.S. Pat. No. 6,357,865 B1 (hereinafter “Kubby '865”);

Joel A. Kubby et al., “Method of fabricating a micro-electro-mechanical fluid ejector,” U.S. Pat. No. 6,662,448 B2 (hereinafter “Kubby '448”);

Nathan S. Lewis et al., “Sensor array for detecting analytes in fluids,” U.S. Pat. No. 5,571,401 (hereinafter “Lewis”);

Edward J. Martens III et al., “Delivery system for dispensing volatiles,” U.S. Pat. No. 6,378,780;

Stephen Montague et al., “Method for integrating microelectromechanical devices with electronic circuitry,” U.S. Pat. No. 5,798,283 (hereinafter “Montague”);

Robert D. Nasby et al., “Use of chemical mechanical polishing in micromachining,” U.S. Pat. No. 5,804,084 (hereinafter “Nasby”);

Eric Peeters et al., “Print head for use in a ballistic aerosol marking apparatus,” U.S. Pat. No. 6,116,718 (hereinafter “Peeters '718”);

Eric Peeters et al., “Ballistic aerosol marking apparatus for marking with a liquid material,” U.S. Pat. No. 6,328,409;

M. Steven Rodgers et al., “Method for fabricating five-level microelectromechanical structures and microelectromechanical transmission formed,” U.S. Pat. No. 6,082,208 (hereinafter “Rodgers”);

Kia Silverbrook, “Method of manufacture of a thermally actuated ink jet including a tapered heater element,” U.S. Pat. No. 6,180,427 (hereinafter “Silverbrook”); and

Scott Eliott, “Security system for video game system with hard disk drive and internet access capability”, U.S. Pat. No. 6,712,704 (hereinafter “Eliott”).

### BACKGROUND OF THE INVENTION

There is a need to provide improved systems to alter the physical characteristics of the atmosphere in human habitable dwellings such as a home, store, or workplace. Systems are well known to alter physical aspects of the ambient atmosphere, such as the temperature, cleanliness or humidity of an atmosphere and to alter circulation properties of an atmosphere within some area or environmental region. There are also systems to dispense airborne materials such as fragrances, perfumes, deodorizers, sanitizers, pheromones and the like. However, current systems do not provide the desired degree of control and flexibility with respect to the amount, time and type of material that is dispensed. It is further desirable to be able to provide this capability at low cost, with a device that operates with a large range of materials, and that can be configured to selectively dispense one or more materials from a set of materials.

Current devices and systems do not provide the desired degree of control and flexibility with respect to the amount, time and type of material that is dispensed.

It is desirable to be able to provide this capability at low cost, with fluid dispensing devices, systems, or both, that are compact in size, operate with a large range of materials, and that can be configured to selectively dispense one or more materials from a set of materials.

Thus, there is a need for an improved environmental system.

### SUMMARY OF THE INVENTION

In a first aspect of the invention, there is described an environmental system including a micromechanical dispensing device, the environmental system comprising an environmental system controller arranged to communicate with one or more included environmental air units, the one or more environmental air units arranged to alter or control one or more physical properties of an atmosphere, the environmental system controller further arranged to communicate with an included micromechanical dispensing device, the micromechanical dispensing device arranged to dispense one or more fluids into the atmosphere, the micromechanical dispensing device comprising one or more micromechanical dispensing mechanisms, each of the one or more micromechanical dispensing mechanisms arranged to fluidly couple to a corresponding fluid reservoir of one or more fluid reservoirs.

In a second aspect of the invention, there is described an environmental system including a micromechanical dispensing device, the environmental system comprising an environmental system controller arranged to communicate with one or more included environmental air units, the one or more environmental air units arranged to alter or control one or more physical properties of an atmosphere, the environmental system controller further arranged to communicate with an included micromechanical dispensing device, the micromechanical dispensing device arranged to dispense a plurality of fluids into the atmosphere, the micromechanical dispensing device comprising a plurality of micromechanical dispensing mechanisms, each of the plurality of micromechanical dispensing mechanisms arranged to fluidly couple to a corresponding fluid reservoir of a plurality of fluid reservoirs.

In a third aspect of the invention, there is described an environmental system including a micromechanical dispensing device, the environmental system comprising an environmental system controller arranged to communicate with one or more included environmental air units, the one or more environmental air units arranged to alter or control one or more physical properties of an atmosphere, the environmental system controller further arranged to communicate with an included micromechanical dispensing device, the micromechanical dispensing device arranged to dispense a plurality of fluids into the atmosphere, the micromechanical dispensing device comprising a micromechanical dispensing mechanism that is fluidly coupled to an included valve, wherein the valve is arranged to selectively fluidly couple the micromechanical dispensing mechanism to a plurality of fluid reservoirs.

In a fourth aspect of the invention, there is described an environmental system including a micromechanical dispensing device, the environmental system comprising an environmental system controller arranged to communicate with one or more included environmental air units, the one or more environmental air units arranged to alter or control one or more physical properties of an atmosphere, the environmental system controller further arranged to communicate with an included micromechanical dispensing device, the micromechanical dispensing device arranged to dispense a fluid into the atmosphere, the micromechanical dispensing device comprising a plurality of micromechanical dispensing mechanisms arranged to fluidly couple to a fluid reservoir.

In a fifth aspect of the invention, there is described an environmental system including a micromechanical dispensing device, the environmental system comprising an environmental system controller arranged to communicate with one or more included environmental air units, the one or more environmental air units arranged to alter or control one or more physical properties of an atmosphere, the environmental system controller further arranged to communicate with an included micromechanical dispensing device, the micromechanical dispensing device arranged to dispense at least one fluid into the atmosphere.

### BRIEF DESCRIPTION OF THE SEVERAL VIEWS OF THE DRAWING

FIG. 1 depicts a typical dispensing device 100 for dispensing a fluid into an atmosphere, as in the prior art.

FIG. 2 depicts one embodiment of a micromechanical dispensing device 200 arranged to dispense one or more fluids into an atmosphere.

FIG. 3 depicts a dispensing system 300 for dispensing one or more fluids into an atmosphere using the FIG. 2 micromechanical dispensing device 200.

FIG. 4 depicts one embodiment of a micromechanical dispensing device 400 to dispense a plurality of fluids into an atmosphere.

FIG. 5 depicts a dispensing system 500 for dispensing a plurality of fluids into an atmosphere using the FIG. 4 micromechanical dispensing device 400.

FIG. 6 depicts another embodiment of a micromechanical dispensing device 600 arranged to dispense a plurality of fluids into an atmosphere.

FIG. 7 depicts one embodiment of a micromechanical dispensing device 700 arranged to dispense a fluid into an atmosphere.

FIG. 8A depicts a first embodiment 800A of an environmental system including a micromechanical dispensing device. As shown, the environmental system 800A uses the FIG. 2 micromechanical dispensing device 200.

FIG. 8B depicts a second embodiment 800B of an environmental system including a micromechanical dispensing device. As shown, the environmental system 800B uses the FIG. 4 micromechanical dispensing device 400.

FIG. 8C depicts a third embodiment 800C of an environmental system including a micromechanical dispensing device. As shown, the environmental system 800C uses the FIG. 6 micromechanical dispensing device 600.

FIG. 8D depicts a fourth embodiment 800D of an environmental system including a micromechanical dispensing device. As shown, the environmental system 800D uses the FIG. 7 micromechanical dispensing device 700.

FIG. 9 depicts a dispensing system 900 using any of the FIG. 6 micromechanical dispensing device 600 and the FIG. 7 micromechanical dispensing device 700.

FIG. 10 depicts a video game system 1000 including a micromechanical dispensing device 1090. As shown, the micromechanical dispensing device 1090, in turn, comprises any of the FIG. 2 micromechanical dispensing device 200, the FIG. 4 micromechanical dispensing device 400, the FIG. 6 micromechanical dispensing device 600 and the FIG. 7 micromechanical dispensing device 700.

### DETAILED DESCRIPTION OF THE INVENTION

Briefly, an environmental system includes a micromechanical dispensing device. The environmental system comprises an environmental system controller arranged to communicate with one or more included environmental air units. The one or more environmental air units are arranged to alter or control one or more physical properties of an atmosphere. The environmental system controller is further arranged to communicate with an included micromechanical dispensing device. The micromechanical dispensing device is arranged to dispense at least one fluid into the atmosphere.

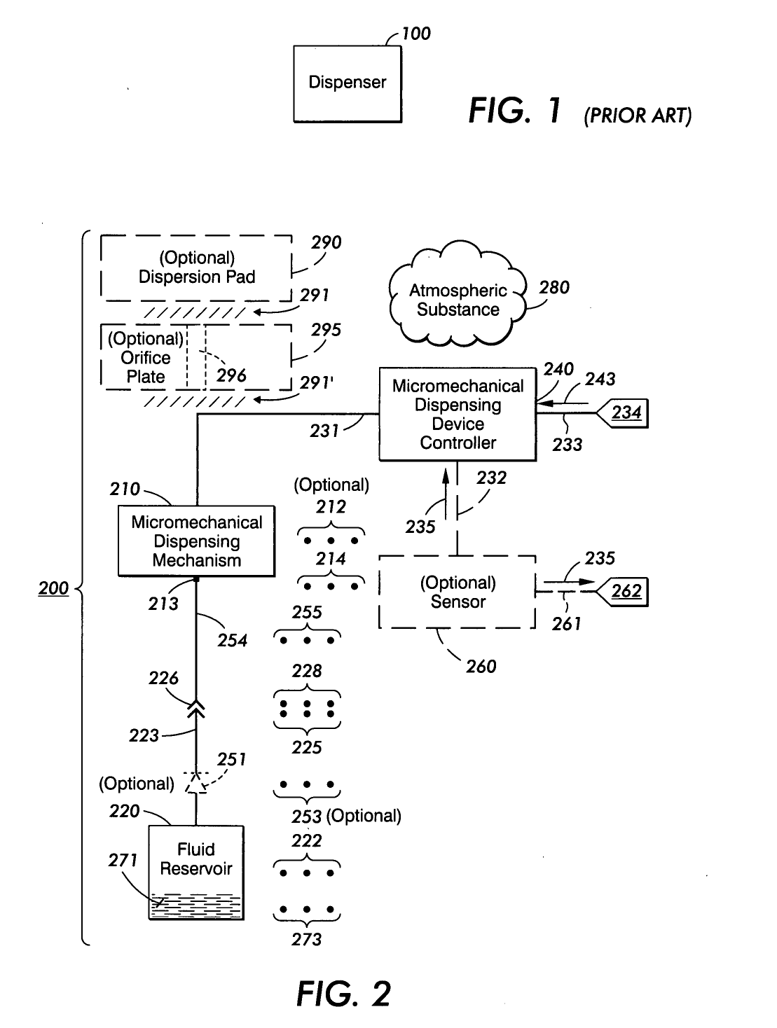

Referring now to FIG. 1, there is depicted a typical dispensing device 100 for dispensing a fluid into an atmosphere, as in the prior art. As is known, typically such devices 100 are controllable by a system controller, as described herein.

Referring now to FIG. 2, there is depicted one embodiment of a micromechanical dispensing device 200 arranged to dispense one or more fluids into an atmosphere.

For good understanding, the term “micromechanical” is sometimes alternately expressed as “micro-electromechanical”. Also, the terms “micromechanical” and “micro-electromechanical” are sometimes abbreviated as “MEMS”.

As shown, the micromechanical dispensing device 200 comprises one or more micromechanical dispensing mechanisms 210, 212 fluidly coupled to a corresponding one or more fluid reservoirs 220, 222.

By a “micromechanical dispensing mechanism”, it is meant a dispensing mechanism formed using micromachining and etching techniques, typically with a silicon-based device, as discussed in greater detail below.

Referring to FIG. 2, the one or more micromechanical dispensing mechanisms 210, 212 include a corresponding one or more inlets 213, 214 for receiving one or more fluids to be dispensed by the mechanisms 210, 212.

The one or more inlets 213, 214, in turn, are fluidly coupled to one or more channels 254, 255.

The one or more channels 254, 255, in turn, are fluidly coupled to one or more channel ports 226, 228.

The one or more channel ports 226, 228, in turn, are arranged to removably interconnect or mate with one or more corresponding fluid reservoir ports 223, 225.

The one or more reservoir ports 223, 225, in turn, are fluidly coupled to the corresponding one or more fluid reservoirs 220, 222.

The one or more fluid reservoirs 220, 222, in turn, contain one or more corresponding fluids 271, 273.

In one embodiment, one or more optional check valves 251, 253 are interposed between the fluid reservoirs 220, 222 and the fluid reservoir ports 223, 225.

As a result of the foregoing arrangement, the one or more fluid reservoirs 220, 222 and the one or more fluids 271, 273 are fluidly coupled to the one or more dispensing mechanisms 210, 212.

One skilled in the art is familiar with a variety of means to construct a removable fluid reservoir. In one embodiment, for example, any of the one or more fluid reservoirs 220, 222 are similar to identical to the fluid reservoir of the Carrese patent.

As shown in FIG. 2, an included dispensing device controller 240 is arranged to actuate or control the one or more micromechanical dispensing mechanisms 210, 212 by means of suitable control signals that are communicated to the dispensing mechanisms 210, 212 by means of a communication link or path 231.

In one embodiment, the device controller 240 comprises any of a number of well-known control and programming electronic circuits or devices well-known to those skilled in the art such as, for example, any of an ASIC, a PGA, a PROM, an EPROM, an EEPROM, an FPGA and a discrete circuit.

In one embodiment, the device controller 240 is comprised of electronic circuitry that is a part of the same micromechanical structure comprising the one or more micromechanical dispensing mechanisms 210, 212.

As shown in FIG. 2, in one embodiment, a program control signal 243 is communicated to the device controller 240 by means of an included controller interface 234 and a communication link or path 233.

Referring still to FIG. 2, in one embodiment, the micromechanical dispensing device 200 further comprises an optional dispensing device sensor 260. The dispensing device sensor 260, in turn, is arranged to form a dispensing device sensor signal 235 based on a concentration of an atmospheric substance 280.

Sensors responsive to the airborne concentration of substances in the atmosphere are well-known to those skilled in the art. For example, the sensor 260 may comprise a sensor similar or identical to the sensor of the Lewis patent.

In one embodiment, the atmospheric substance 280 comprises any of the one or more fluids 271, 273 that are dispensed by the dispensing device 200.

As shown, in one embodiment, the dispensing device sensor 260 is arranged to communicate the dispensing device sensor signal 235 to the controller 240 by means of a communication link or path 232. In turn, the device controller 240 is arranged to actuate or control the one or more of the dispensing mechanisms 210, 212 based at least in part on the dispensing device sensor signal 235.

In another embodiment, the dispensing device sensor signal 235 is communicated to an included dispensing device sensor interface 262 by means of a communication link or path 261.

Still referring to FIG. 2, in one embodiment, the micromechanical dispensing device 200 comprises a dispersion pad 290 positioned to receive a fluid dispensed by the one or more micromechanical dispensing mechanisms 210, 212.

In one embodiment, the dispersion pad 290 comprises any natural or synthetic material or polymer, fiber or strand, either singular or woven, twisted, braided, bundled, molded or shaped in a manner that transports fluid or vapors by capillary action or that can serve as a support medium for the fluid or vapors.

The dispersion pad 290 is separated from the micromechanical dispensing device 200 by a gap 291-291′.

In one embodiment, the gap 291-291′ is minimized to achieve substantially zero distance, providing intimate contact between the dispersion pad 290 and the micromechanical dispensing device 200.

Also depicted in FIG. 2 is an optional orifice plate 295 including an orifice 296. The orifice plate 295 is arranged such that fluid dispensed by the dispensing mechanisms 210, 212 is further dispensed through the orifice 296.

Referring generally to FIG. 2, it will be understood that there are numerous fluids suitable for use with the micromechanical dispensing device 200 to control the quality or other aspects of the atmosphere for aesthetic, hygienic or mood-enhancing effects.

In one embodiment, the dispensing device 200 is arranged to dispense any of the following fluids: fragrance, perfume, therapeutic, mood-enhancing agent, pheromone, moisturizer, humectant, miticide, deodorizer, disinfectant, sanitizing agent and insecticide.

In one embodiment, any of the one or more fluid reservoirs 220, 222 contains a fluid 271, 273 that comprises any of a fragrance, perfume, therapeutic, mood-enhancing agent, pheromone, moisturizer, humectant, miticide, deodorizer, disinfectant, sanitizing agent and insecticide.

As is known, a miticide is one of the known materials to kill mites.

Referring still to FIG. 2, several embodiments of the one or more micromechanical dispensing mechanisms 210, 212 are now described.

In one embodiment, any of the one or more micromechanical dispensing mechanisms 210, 212 comprise an electrostatically-driven membrane. For example, in one embodiment, any of the one or more micromechanical dispensing mechanisms 210, 212 comprise a membrane similar or identical to the electrostatically-actuated diaphragm 10 of the fluid ejector 100 of the Kubby '865 patent.

Referring now to Kubby '865, FIG. 1 discloses a micro-electromechanical fluid ejector 100 fabricated in a standard polysilicon surface micromachining process. depicted in FIG. 1 and described from col. 2, line 65 to col. 3, line 21, the fluid drop ejector 100 comprises a substrate 20, a silicon wafer, an insulator 30, a thin film of silicon nitride, Si3N4, a conductor 40, acting as the counterelectrode, made of metal or a doped semiconductor such as polysilicon, and a membrane 50, made from polysilicon as is typically used in a surface micromachining process.

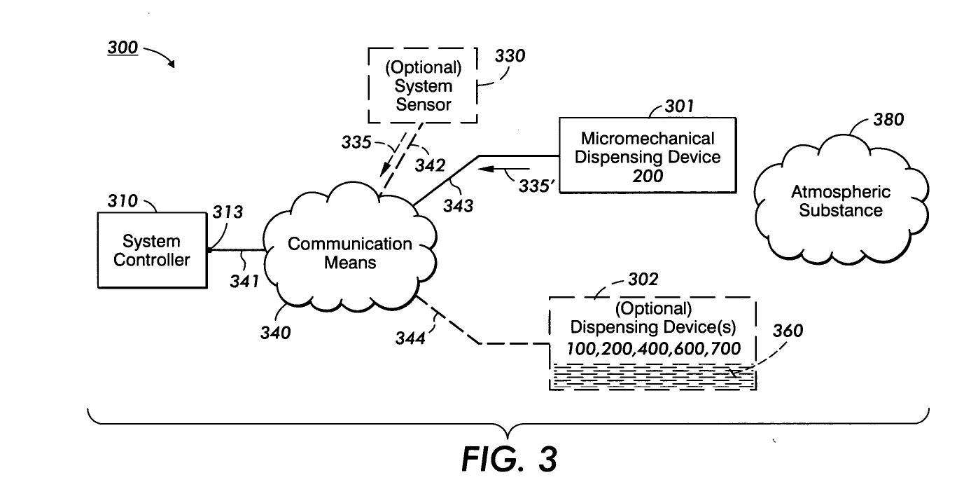

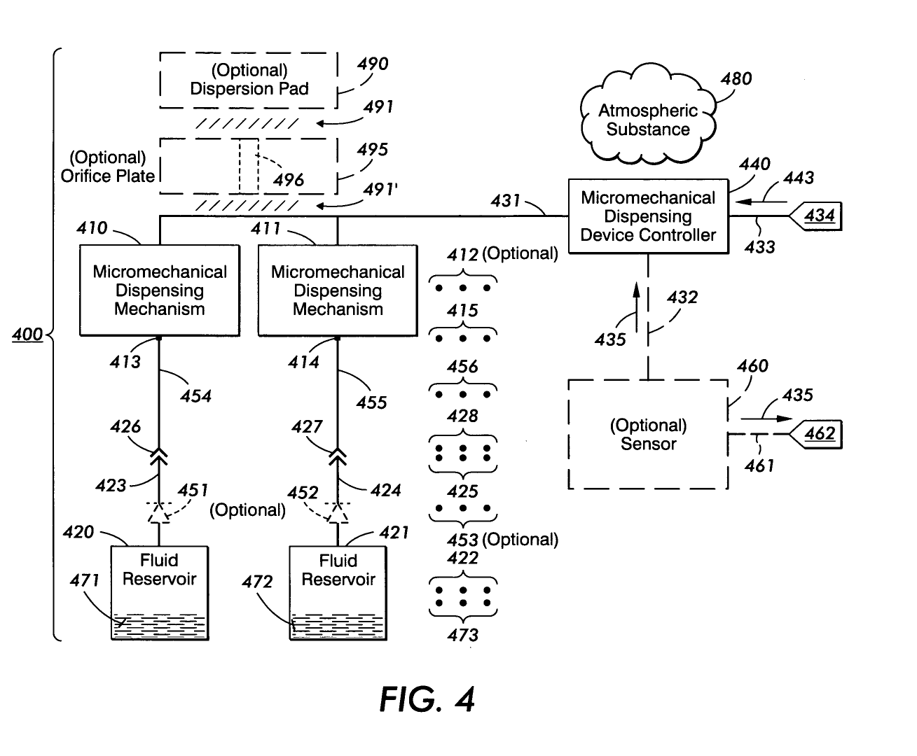

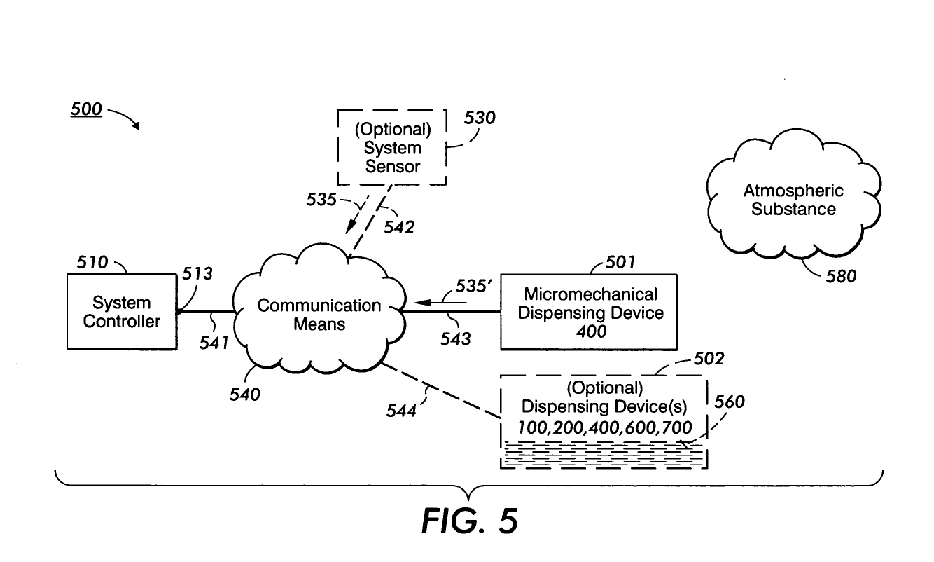

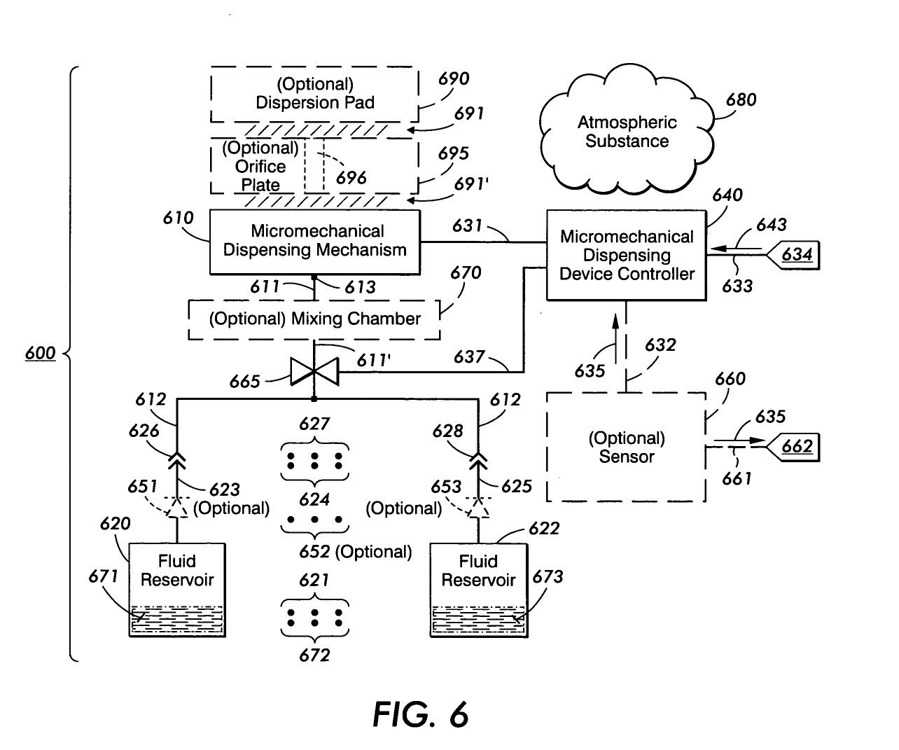

Still referring to Kubby '865, the operation of the micromechanical dispensing mechanism 100 is described from col. 2, line 65 to col. 4, line 27. As described therein, a power source, element P, shown in FIG. 1, is applied between the membrane 10 and the conductor 40 to cause displacement of the membrane 10. The patent's FIG. 2 shows a cross-section of the displaced membrane 10. As shown in FIG. 4, displacement of the membrane 10 toward the conductor 40 increases the volume of the chamber 70 formed by the membrane 10 enclosed by orifice plate 60. Fluid is thus drawn into the chamber from a fluid reservoir, as described at col. 3, lines 45-46. As shown in FIG. 3, an included nipple 52 serves to limit the displacement of the membrane toward the conductor 40. As shown in FIGS. 5-6, as the voltage between the conductor and the membrane is relaxed, the membrane returns to its initial position, thus creating an increased fluid pressure which ejects a drop of fluid 72.

Still referring to Kubby '865, the process for forming the micromechanical dispensing mechanism 100 is described from col. 6, line 4 to col. 7, line 24.

Referring again to the present FIG. 2, in a further embodiment, any of the one or more micromechanical dispensing mechanisms 210, 212 comprise an electrostatically-actuated piston. For example, in one embodiment, any of the one or more micromechanical dispensing mechanisms 210, 212 comprise a piston similar or identical to the electrostatically-actuated piston 110 of the fluid ejector 100 of the Gooray '915 patent.

Referring now to Gooray '915, FIG. 1 discloses a micromechanical fluid ejector 100 fabricated using a “SUMMiT” processes or other suitable micromachining processes. As described at col. 3, lines 14-21, the SUMMiT processes are described in various U.S. patents, including the aforementioned patents Fariono, Montague, Nasby, Barron '548, Barron '788 and Rodgers. As depicted in FIG. 1 and described at col. 4, lines 35-65 the fluid drop ejector 100 comprises a movable piston structure 110, a stationary face plate 130, a fluid chamber 120 and a substrate 150.

In one embodiment, the piston structure 110 is resiliently mounted on the substrate 150 by one or more spring elements 114. The stationary face plate 130 further includes a nozzle hole 132 through which a fluid drop is ejected.

Still referring to Gooray '915, the piston structure 110 moves towards the faceplate 130 due to electrostatic attraction between the piston structure 110 and the faceplate 130, ejecting fluid through nozzle hole 132, as described at col. 2, lines 51-54. Further embodiments of an electrostatically-driven piston are described from col. 4, line 66 to col. 6, line 53.

Again referring to the present FIG. 2, in another embodiment, any of the one or more micromechanical dispensing mechanisms 210, 212 comprise a magnetically-actuated membrane. For example, in one embodiment, any of the one or more micromechanical dispensing mechanisms 210, 212 comprise a membrane similar or identical to the magnetically-actuated diaphragm 38 of the fluid ejector 12 of the Genovese patent.

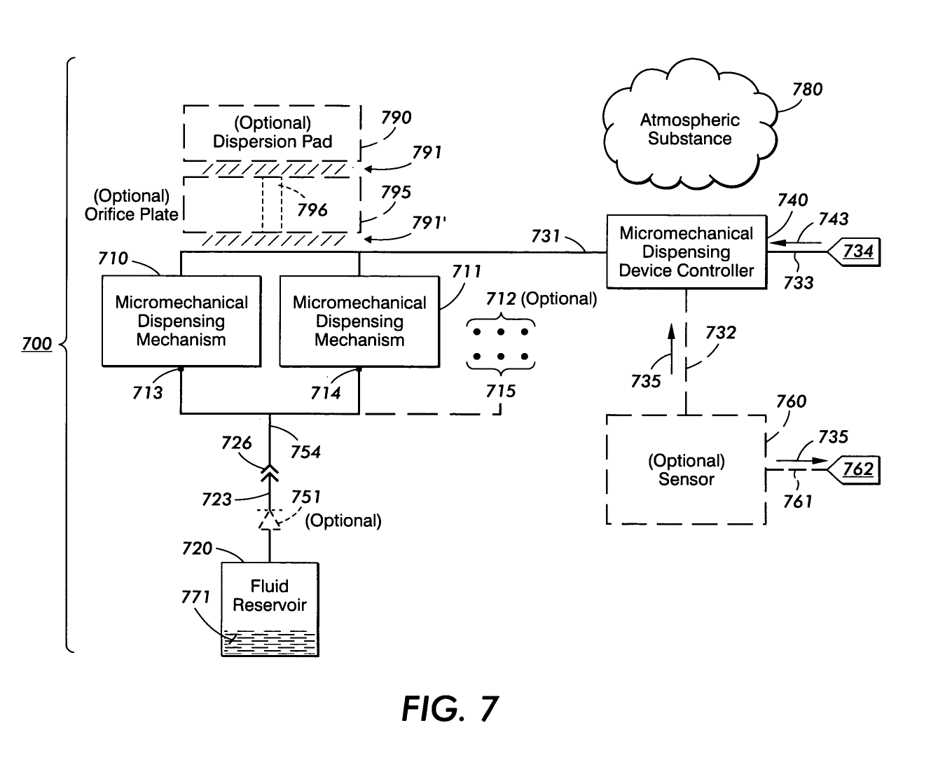

Referring now to Genovese, a micro-electromechanical fluid ejector 12 is depicted in FIG. 7. As described at col. 5, lines 9-40, the fluid drop ejector 12 comprises a silicon plate 32, including two parallel surfaces 33, 34, with a thickness of about 20 mils (0.020 inches) or approximately 500 microns. The silicon plate 32 is anisotropically etched from the surface 34 to form a recess 36 and form a membrane 38 for use as a diaphragm. The diaphragm 38, with a bottom surface 37 is preferably about 1 micron in thickness.

Still referring to Genovese, as described at col. 5, lines 16-19, alternately, a plate of silicon or ceramic is used in conjunction with an appropriate process such as molding or laser ablation. The silicon top surface 33 has an electrode 40 deposited onto it such that at least a portion of the electrode 40 lies on top of diaphragm 38. An orifice plate 44 with internal cavity 49, and aligned with diaphragm 38 is formed on silicon surface 33. As described at col. 5, lines 35-40, the internal cavity 49 is filled with fluid.

Referring still to Genovese, the operation of the magnetically-actuated diaphragm is described at col. 5, lines 41-67. The fluid ejector is subject to a predetermined magnetic field B with a field direction extending upward with respect to FIG. 7, the upwards direction corresponding to a direction approximately perpendicular to surface 33 and electrode 40. As the result of the selective application of electric current pulses from left to right through the electrode 40 (as in FIG. 7), a Force F is generated which deforms the diaphragm 38 in the upward direction towards the nozzle. As described at col. 5, lines 50-59, this application of pulses results in ejection of drops from the nozzle, with drop volume determined by the electric current pulses.

Still referring to Genovese, the process for forming the micromechanical dispensing mechanism is described from col. 7, line 13 to col. 8, line 51.

Referring again to the present FIG. 2, in another embodiment, any of the one or more micromechanical dispensing mechanisms 210, 212 comprise a ballistic aerosol micromechanical dispensing mechanism. For example, in one embodiment, any of the one or more micromechanical dispensing mechanisms 210, 212 are similar or identical to the aerosol ballistic dispensing device 24 of the Peeters '718 patent.

Referring now to Peeters '718, there is described from col. 6, line 66 to col. 7, line 28 a ballistic aerosol dispensing device 24 particularly adapted for deposition of materials onto a substrate for printing. The ballistic aerosol dispensing device comprises a body 26 within which is formed a plurality of cavities 28 for receiving materials to be dispensed on a surface. Also formed in body 26 may be a propellant cavity 30. Fitting 32 may be provided for connecting cavity 30 to a propellant source 33 such as a compressor, a propellant reservoir or the like. Body 26 may be connected to a print head 34 that will be discussed later. As depicted in FIG. 3 and described at col. 7, lines 29-40, the cavities 28 further comprise ports 42, which provide communication between cavities 28 and a channel 46. In a similar manner, as described with reference to FIG. 3 and described at col. 8, lines 41-65, cavity 30 includes a port 44 providing communication between the cavity and channel 46 through which propellant may travel.

Still referring to Peeters '718, the operation of a ballistic aerosol dispensing device is described from col. 8, line 48 to col. 9, line 6. As discussed, propellant enters the channel 46 through port 44, from the propellant cavity 30. The propellant flows continuously through the channel while the dispensing apparatus is operative, or else is modulated such that the propellant passes through the channel only when material is to be dispensed. Such propellant modification may be accomplished by a valve 31 interposed between the propellant source 33 and the channel 46. Material may controllably enter the channel 46 through one or more of the ports 42.

Referring still to Peeters '718, one embodiment of a process for forming a micromechanical dispensing mechanism incorporating a ballistic aerosol mechanism is described from col. 9, line 7 to col. 10, line 7.

Again referring to the present FIG. 2, in another embodiment, any of the one or more micromechanical dispensing mechanisms 210, 212 comprise an arrangement incorporating a thermally-actuated paddle vane. For example, in one embodiment, any of the one or more micromechanical dispensing mechanisms 210, 212 comprise an arrangement of a thermally-actuated paddle vane similar or identical to the fluid ejector 20 of the Silverbrook patent.

Referring now to Silverbrook, there is described from col. 9, line 58 to col. 10, line 60 a nozzle arrangement comprising a thermally-actuated paddle vane for dispensing fluids, the nozzle arrangement formed using standard micro-electromechanical techniques. The nozzle arrangement comprises an actuator arm 21 which includes a bottom arm 22, constructed from a conductive material such as a copper nickel alloy, and a top layer 25 composed from the same material. The layer 22 includes a tapered end portion near the end post 24. The layer 22 is connected to the lower CMOS layers 26, which are formed in the standard manner on a silicon substrate surface 27. The tapering of layer 22 means that any conductive resistive heating occurs near the post portion 24. The actuator arm 21 is interconnected to an ejection paddle located within a nozzle chamber 28. The nozzle chamber includes an ejection nozzle 29 from which ink is ejected. The nozzle further includes a slot arrangement 30, which results in minimum fluid outflow through the actuator arm interconnection and also results in minimal pressure increases in this area. An ink supply channel 39 is provided by back etching through the wafer to the back surface of the nozzle.

Still referring to Silverbrook, the operation of a fluid micromechanical dispensing mechanism based on a thermally-actuated paddle vane is described at col. 9, lines 10-57, with reference to FIGS. 2-3. Inside nozzle chamber 2, a paddle type device 7 is interconnected to an actuator arm 8 through a slot in the wall of nozzle chamber 2. The actuator arm includes a heater means 9 located adjacent to a post end portion 20, the post end affixed to a substrate. To eject a drop, heater means 9 is heated so as to undergo thermal expansion. Ideally, the heater means is located adjacent to the post end portion 20 such that the effects of activation result in large movements of the paddle end 7. Upon heating, the heating means 9 undergoes thermal expansion, resulting in a general increase in pressure around the meniscus 5. The heater current is pulsed and fluid is ejected out of the nozzle 4 in addition to flowing in from the fluid channel 3. Subsequently, the paddle 7 is deactivated to return to its quiescent position.

Referring still to Silverbrook, a process for forming a fluid micromechanical dispensing mechanism that comprises a thermally-actuated paddle vane using standard micro-electromechanical techniques from col. 10, line 64 to col. 13, line 41.

Referring generally to the present FIG. 2, it is believed that all information, know-how and resources needed to enable the various communications between and amongst the components depicted therein and described above in connection therewith are common and well-known to those skilled in the art.

Still referring generally to the present FIG. 2, in one embodiment, any of the one or more micromechanical dispensing mechanisms 210, 212 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 3, there is depicted a dispensing system 300 for dispensing one or more fluids into an atmosphere. As shown, the dispensing system 300 comprises a dispensing device 301. The dispensing device 301, in turn, comprises the micromechanical dispensing device 200 described in connection with FIG. 2 above.

With momentary cross-reference to FIG. 2, in one embodiment the dispensing device 301 (corresponding to the dispensing device 200 in FIG. 2) is arranged to dispense only one (1) fluid and thus the dispensing device 301 comprises only the micromechanical dispensing mechanism 210 arranged to dispense the fluid 271.

With continued momentary cross-reference to FIG. 2, in another embodiment the dispensing device 301 (corresponding to the dispensing device 200 in FIG. 2) is arranged to dispense a plurality of fluids and thus the dispensing device 301 comprises a plurality of micromechanical dispensing mechanisms 210, 212 arranged to dispense the plurality of fluids 271, 273.

Still referring to FIG. 3, in one embodiment the dispensing system 300 comprises only a single dispensing device, namely, the dispensing device 301.

In another embodiment, the dispensing system 300 comprises the dispensing device 301 and, in addition, the dispensing system 300 further comprises at least one additional dispensing device depicted as element 302 in FIG. 3. In turn, the at least one additional dispensing device 302 comprises any of the dispensing devices 100, 200, 400, 600 and 700.

For good understanding, the dispensing devices 100 and 200 in FIG. 3 correspond to the dispensing devices 100 and 200 described above in connection with FIGS. 1 and 2. Also, the dispensing devices 400, 600 and 700 in FIG. 3 correspond to the dispensing devices 400, 600 and 700 described below in connection with FIGS. 4, 6 and 7.

Referring still to FIG. 3, when the dispensing device 301 and the at least one additional dispensing device 302 are both present, the dispensing device 301 dispenses one or more fluids 271, 273 and the at least one additional dispensing device 302 dispenses one or more fluids depicted as reference number 360 in FIG. 3.

Thus, in general, it will be understood that in various embodiments the dispensing system 300 is capable of dispensing a wide variety of combinations and permutations of fluids.

As shown in FIG. 3, the dispensing system 300 further comprises a dispensing system controller 310. The dispensing system controller 310, in turn, comprises a controller communication interface 313. The dispensing system controller 310 is arranged to actuate or control the dispensing device 301 by means of suitable control signals that are communicated to the dispensing device 301 by means of the controller communication interface 313, a communication link or path 341, a communication means 340 and a communication link or path 343.

With momentary cross-reference to FIG. 2, the suitable control signals described in connection with FIG. 3 above correspond to the program control signal 243 in FIG. 2. As described in connection with FIG. 2, the program control signal 243 is communicated to the device controller 240 comprised in the dispensing device 200 by means of the included controller interface 234 and the communication link or path 233.

Referring again to FIG. 3, in one embodiment the system controller 310 is further arranged to actuate or control the optional at least one additional dispensing device 302 by means of suitable control signals that are communicated by means of the link or path 341, the communication means 340 and a communication link or path 344.

Still referring to FIG. 3, in one embodiment an optional system sensor 330 is provided. For example, the system sensor 330 may be similar or identical to the sensor of the Lewis patent.

As shown, the system sensor 330 is arranged to form a system sensor signal 335 based on a concentration of an atmospheric substance 380 and to communicate the system sensor signal 335 to the system controller 310 by means of a communication link or path 342, the communication means 340 and the link or path 341.

With cross-reference to FIG. 2, in another embodiment the dispensing device 301 further comprises the optional dispensing device sensor 260 that is described in connection with FIG. 2. As shown in the present FIG. 3, in this latter embodiment the dispensing device sensor 260 of the dispensing device 301 is arranged to form a system sensor signal 335′ (corresponding to the dispensing device sensor signal 235 as communicated to the dispensing device sensor interface 262 in FIG. 2) based on the atmospheric substance 380 and to communicate the system sensor signal 335′ to the system controller 310 by means of the link or path 343, the communication means 340 and the link or path 341.

Still referring to FIG. 3, in one embodiment the system controller 310 is arranged to actuate or control any of the dispensing devices 301 and 302 based at least in part on the system sensor signal 335 that is formed by the system sensor 330.

In another embodiment, the system controller 310 is arranged to actuate or control any of the dispensing devices 301 and 302 based at least in part on the system sensor signal 335′ that is formed by the dispensing device sensor 260 of the dispensing device 301.

In one embodiment, the communication means 340 and the communication links or paths 341, 342, 343 and 344 comprise a communication network.

In one embodiment, the communication means 340 and the links or paths 341, 342, 343 and 344 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 3, it is believed that all information, know-how and resources needed to enable the various communications between and amongst the components depicted therein and described above in connection therewith are common and well-known to those skilled in the art.

Referring now to FIG. 4, there is depicted one embodiment of a micromechanical dispensing device 400 to dispense a plurality of fluids into an atmosphere.

As shown, the micromechanical dispensing device 400 comprises a plurality of micromechanical dispensing mechanisms 410, 411, 412 fluidly coupled to a corresponding plurality of fluid reservoirs 420, 421, 422.

As shown, the plurality of dispensing mechanisms 410, 411, 412 include a corresponding plurality of inlets 413, 414, 415 for receiving fluids to be dispensed by the mechanisms 410, 411, 412.

The plurality of inlets 413, 414, 415, in turn, are fluidly coupled to a corresponding plurality of channels 454, 455, 456.

The plurality of channels 454, 455, 456, in turn, are fluidly coupled to a corresponding plurality of channel ports 426, 427, 428.

The plurality of channel ports 426, 427, 428, in turn, are arranged to removably interconnect or mate with a corresponding plurality of fluid reservoir ports 423, 424, 425.

The plurality of fluid reservoir ports 423, 424, 425, in turn, are fluidly coupled to the corresponding plurality of fluid reservoirs 420, 421, 422.

The plurality of fluid reservoirs 420, 421, 422, in turn, contain a corresponding plurality of fluids 471, 472, 473.

In one embodiment, an optional plurality of check valves 451, 452, 453 are interposed between the plurality of fluid reservoirs 420, 421, 422 and the plurality of fluid reservoir ports 423, 424, 425.

As a result of the foregoing arrangement, the plurality of fluid reservoirs 420, 421, 422 and the plurality of fluids 471, 472, 473 are fluidly coupled to the plurality of dispensing mechanisms 410, 411, 412.

One skilled in the art is familiar with a variety of means to construct a removable fluid reservoir. In one embodiment, for example, any of the plurality of fluid reservoirs 420, 421, 422 are similar or identical to the fluid reservoir of the Carrese patent.

As shown in FIG. 4, an included dispensing device controller 440 is arranged to actuate or control the plurality of micromechanical dispensing mechanisms 410, 411, 412 by means of suitable control signals that are communicated to the dispensing mechanisms 410, 411, 412 by means of a communication link or path 431.

In one embodiment, the device controller 440 comprises any of a number of well-known control and programming electronic circuits or devices well-known to those skilled in the art such as, for example, any of an ASIC, a PGA, a PROM, an EPROM, an EEPROM, an FPGA and a discrete circuit.

In one embodiment, the device controller 440 is comprised of electronic circuitry that is a part of the same micromechanical structure comprising the micromechanical dispensing mechanisms 410, 411, 412.

As shown in FIG. 4, in one embodiment, a program control signal 443 is communicated to the device controller 440 by means of an included controller interface 434 and a communication link or path 433.

Referring still to FIG. 4, in one embodiment, the micromechanical dispensing device 400 further comprises an optional dispensing device sensor 460. The dispensing device sensor 460, in turn, is arranged to form a dispensing device sensor signal 435 based on a concentration of an atmospheric substance 480.

Sensors responsive to the airborne concentration of substances in the atmosphere are well-known to those skilled in the art. For example, the sensor 260 may comprise a sensor similar or identical to the sensor of the Lewis patent.

In one embodiment, the atmospheric substance 480 comprises any of the fluids 471, 472, 473 that are dispensed by the dispensing device 400.

As shown, in one embodiment, the dispensing device sensor 460 is arranged to communicate the dispensing device sensor signal 435 to the controller 440 by means of a communication link or path 432. In turn, the device controller 440 is arranged to actuate or control the dispensing mechanisms 410, 411, 412 based at least in part on the dispensing device sensor signal 435.

In another embodiment, the dispensing device sensor signal 435 is communicated to an included dispensing device sensor interface 462 by means of a communication link or path 461.

Still referring to FIG. 4, in one embodiment, the micromechanical dispensing device 400 comprises a dispersion pad 490 positioned to receive a fluid dispensed by the micromechanical dispensing mechanisms 410, 411, 412.

In one embodiment, the dispersion pad 490 comprises any natural or synthetic material or polymer, fiber or strand, either singular or woven, twisted, braided, bundled, molded or shaped in a manner that transports fluid or vapors by capillary action or that can serve as a support medium for the fluid or vapors.

The dispersion pad 490 is separated from the micromechanical dispensing device 400 by a gap 491-491′.

In one embodiment, the gap 491-491′ is minimized to achieve substantially zero distance, providing intimate contact between the dispersion pad 490 and the dispensing device 200.

Also depicted in FIG. 4 is an optional orifice plate 495 including an orifice 496. The orifice plate 495 is arranged such that fluid dispensed by the dispensing mechanisms 410, 411, 412 is further dispensed through the orifice 496.

Referring generally to FIG. 4, it will be understood that there are numerous fluids suitable for use with the micromechanical dispensing device 400 to control the quality or other aspects of the atmosphere for aesthetic, hygienic or mood-enhancing effects.

In one embodiment, the dispensing device 400 is arranged to dispense any of the following fluids: fragrance, perfume, therapeutic, mood-enhancing agent, pheromone, moisturizer, humectant, miticide, deodorizer, disinfectant, sanitizing agent and insecticide.

In one embodiment, any of the plurality of fluid reservoirs 420, 421, 422 contains a fluid 471, 472, 473 that comprises any of a fragrance, perfume, therapeutic, mood-enhancing agent, pheromone, moisturizer, humectant, miticide, deodorizer, disinfectant, sanitizing agent and insecticide.

In one embodiment, any of the plurality of micromechanical dispensing mechanisms 410, 411, 412 comprise an electrostatically-driven membrane similar or identical to the electrostatically-driven membrane of the Kubby '865 patent.

In another embodiment, any of the plurality of micromechanical dispensing mechanisms 410, 411, 412 comprise an electrostatically-actuated piston similar or identical to the electrostatically-actuated piston of the Gooray '915 patent.

In a further embodiment, any of the plurality of micromechanical dispensing mechanisms 410, 411, 412 comprise a magnetically-actuated membrane similar or identical to the magnetically-actuated membrane of the Genovese patent.

In still another embodiment, any of the plurality of micromechanical dispensing mechanisms 410, 411, 412 comprise a ballistic aerosol dispensing mechanism similar or identical to the ballistic aerosol dispensing mechanism of the Peeters '718 patent.

In a still further embodiment, any of the plurality of micromechanical dispensing mechanisms 410, 411, 412 comprise a thermally-actuated paddle vane similar or identical to the thermally-actuated paddle-vane of the Silverbrook patent.

Referring generally to the present FIG. 4, it is believed that all information, know-how and resources needed to enable the various communications between and amongst the components depicted therein and described above in connection therewith are common and well-known to those skilled in the art.

Still referring generally to the present FIG. 4, in one embodiment, any of the plurality of micromechanical dispensing mechanisms 410, 411, 412 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 5, there is depicted a dispensing system 500 for dispensing one or more fluids into an atmosphere. As shown, the dispensing system 500 comprises a dispensing device 501. The dispensing device 501, in turn, comprises the micromechanical dispensing device 400 described in connection with FIG. 4 above.

With momentary cross-reference to FIG. 4, the dispensing device 501 (corresponding to the dispensing device 400 in FIG. 4) comprises a plurality of micromechanical dispensing mechanisms 410, 411, 412 arranged to dispense a plurality of fluids 471, 472, 473.

Still referring to FIG. 5, in one embodiment the dispensing system 500 comprises only a single dispensing device, namely, the dispensing device 501.

In another embodiment, the dispensing system 500 comprises the dispensing device 501 and, in addition, the dispensing system 500 further comprises at least one additional dispensing device depicted as element 502 in FIG. 5. In turn, the at least one additional dispensing device 502 comprises any of the dispensing devices 100, 200, 400, 600 and 700.

For good understanding, the dispensing devices 100, 200 and 400 in FIG. 5 correspond to the dispensing devices 100, 200 and 400 described above in connection with FIGS. 1, 2 and 4. Also, the dispensing devices 600 and 700 in FIG. 5 correspond to the dispensing devices 600 and 700 described below in connection with FIGS. 6 and 7.

Referring still to FIG. 5, when the dispensing device 501 and the at least one additional dispensing device 502 are both present, the dispensing device 501 dispenses a plurality of fluids 471, 472, 473 and the at least one additional dispensing device 502 dispenses one or more fluids depicted as reference number 560 in FIG. 5.

Thus, in general, it will be understood that in various embodiments the dispensing system 500 is capable of dispensing a wide variety of combinations and permutations of fluids.

As shown in FIG. 5, the dispensing system 500 further comprises a dispensing system controller 510. The dispensing system controller 510, in turn, comprises a controller communication interface 513. The dispensing system controller 510 is arranged to actuate or control the dispensing device 501 by means of suitable control signals that are communicated to the dispensing device 501 by means of the controller communication interface 513, a communication link or path 541, a communication means 540 and a communication link or path 543.

With momentary cross-reference to FIG. 4, the suitable control signals described in connection with FIG. 5 above correspond to the program control signal 443 in FIG. 4. As described in connection with FIG. 4, the program control signal 443 is communicated to the device controller 440 comprised in the dispensing device 400 by means of the included controller interface 434 and the communication link or path 433.

Referring again to FIG. 5, in one embodiment the system controller 510 is further arranged to actuate or control the optional at least one additional dispensing device 502 by means of suitable control signals that are communicated by means of the link or path 541, the communication means 540 and a communication link or path 544.

Still referring to FIG. 5, in one embodiment an optional system sensor 530 is provided. For example, the system sensor 530 may be similar or identical to the sensor of the Lewis patent.

As shown, the system sensor 530 is arranged to form a system sensor signal 535 based on a concentration of an atmospheric substance 580 and to communicate the system sensor signal 535 to the system controller 510 by means of a communication link or path 542, the communication means 540 and the link or path 541.

With cross-reference to FIG. 4, in another embodiment the dispensing device 501 further comprises the optional dispensing device sensor 460 that is described in connection with FIG. 4. As shown in the present FIG. 5, in this latter embodiment the dispensing device sensor 460 of the dispensing device 501 is arranged to form a system sensor signal 535′ (corresponding to the dispensing device sensor signal 435 as communicated to the dispensing device sensor interface 462 in FIG. 4) based on the atmospheric substance 580 and to communicate the system sensor signal 535′ to the system controller 510 by means of the link or path 543, the communication means 540 and the link or path 541.

Still referring to FIG. 5, in one embodiment the system controller 510 is arranged to actuate or control any of the dispensing devices 501 and 502 based at least in part on the system sensor signal 535 that is formed by the system sensor 530.

In another embodiment, the system controller 510 is arranged to actuate or control any of the dispensing devices 501 and 502 based at least in part on the system sensor signal 535′ that is formed by the dispensing device sensor 460 of the dispensing device 501.

In one embodiment, the communication means 540 and the communication links or paths 541, 542, 543 and 544 comprise a communication network.

In one embodiment, the communication means 540 and the links or paths 541, 542, 543 and 544 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 5, it is believed that all information, know-how and resources needed to enable the various communications between and amongst the components depicted therein and described above in connection therewith are common and well-known to those skilled in the art.

Referring now to FIG. 6, there is depicted another embodiment of a micromechanical dispensing device 600 arranged to dispense a plurality of fluids into an atmosphere.

As discussed below, the dispensing device 600 comprises a micromechanical dispensing mechanism 610 arranged with a valve 665 to selectively fluidly couple the dispensing mechanism 610 to a plurality of fluid reservoirs 620, 621, 622.

As shown, the dispensing mechanism 610 includes an inlet 613 for receiving fluids to be dispensed by the mechanism 610.

The inlet 613, in turn, is coupled to a channel 611-611′.

The channel 611-611′, in turn, is fluidly coupled to a first (output) port of the valve 665.

A second (input) port of the valve 665, in turn, is coupled to a channel 612.

The channel 612, in turn, is fluidly coupled to a plurality of channel ports 626, 627, 628.

The plurality of channel ports 626, 627, 628, in turn, are arranged to removably interconnect or mate with a corresponding plurality of fluid reservoir ports 623, 624, 625.

The plurality of fluid reservoir ports 623, 624, 625, in turn, are fluidly coupled to the corresponding plurality of fluid reservoirs 620, 621, 622.

The plurality of fluid reservoirs 620, 621, 622, in turn, contain a corresponding plurality of fluids 671, 672, 673.

In one embodiment, an optional plurality of check valves 651, 652, 653 are interposed between the fluid reservoirs 620, 621, 622 and the fluid reservoir ports 623, 624, 625.

Referring again to the channel 611-611′, the channel 611-611′ is depicted as comprising a first element 611 and a second element 611′. In one embodiment, an optional mixing chamber 670 to combine fluids is interposed between the channel elements 611 and 611′.

As a result of the foregoing arrangement, the fluid reservoirs 620, 621, 622 and the fluids 671, 672, 673 are fluidly coupled to the valve 665 which, in turn, is fluidly coupled to the dispensing mechanism 610.

Valves for micromechanical systems are well-known to those skilled in the art. In one embodiment, for example, the valve 665 comprises a device similar or identical to the valve of the Cho patent.

One skilled in the art is familiar with a variety of means to construct a removable fluid reservoir. In one embodiment, for example, any of the plurality of fluid reservoirs 620, 621, 622 are similar or identical to the fluid reservoir of the Carrese patent.

As shown in FIG. 6, an included dispensing device controller 640 is arranged to actuate or control the micromechanical dispensing mechanism 610 by means of suitable control signals that are communicated to the dispensing mechanism 610 by means of a communication link or path 631.

As shown, the device controller 640 is further arranged to actuate or control the valve 665 by means of further suitable control signals that are communicated to the valve 665 by means of a communication link or path 637.

Based on the control signals that are communicated by the links or paths 631 and 637, the device controller 640 actuates or controls the valve 665. As a result of such actuating or control by the device controller 640, the valve 665 acts to thereby selectively fluidly couple any of the plurality of fluid reservoirs 620, 621, 622 and the corresponding plurality of fluids 671, 672, 673 to the dispensing mechanism 610 by means of the channels 612 and 611-611′.

In one embodiment, the device controller 640 comprises any of a number of well-known control and programming electronic circuits or devices well-known to those skilled in the art such as, for example, any of an ASIC, a PGA, a PROM, an EPROM, an EEPROM, an FPGA and a discrete circuit.

In one embodiment, the device controller 640 is comprised of electronic circuitry that is a part of the same micromechanical structure comprising the micromechanical dispensing mechanism 610.

As shown in FIG. 6, in one embodiment, a program control signal 643 is communicated to the device controller 640 by means of an included controller interface 634 and a communication link or path 633.

Referring still to FIG. 6, in one embodiment, the micromechanical dispensing device 600 further comprises an optional dispensing device sensor 660. The dispensing device sensor 660, in turn, is arranged to form a dispensing device sensor signal 635 based on a concentration of an atmospheric substance 680.

Sensors responsive to the airborne concentration of substances in the atmosphere are well-known to those skilled in the art. For example, the sensor 660 may comprise a sensor similar or identical to the sensor of the Lewis patent.

In one embodiment, the atmospheric substance 680 comprises any of the fluids 671, 672, 673 that are dispensed by the dispensing device 600.

In one embodiment, the dispensing device sensor 660 is arranged to communicate the dispensing device sensor signal 635 to the controller 640 by means of a communication link or path 632. In turn, the device controller 640 is arranged to actuate or control any of the dispensing mechanism 610 and the valve 665 based at least in part on the dispensing device sensor signal 635.

In another embodiment, the dispensing device sensor signal 635 is communicated to an included dispensing device sensor interface 662 by means of a communication link or path 661.

Still referring to FIG. 6, in one embodiment, the micromechanical dispensing device 600 comprises a dispersion pad 690 positioned to receive a fluid that is dispensed by the micromechanical dispensing mechanism 610.

In one embodiment, the dispersion pad 690 comprises any natural or synthetic material or polymer, fiber or strand, either singular or woven, twisted, braided, bundled, molded or shaped in a manner that transports fluid or vapors by capillary action or that can serve as a support medium for the fluid or vapors.

The dispersion pad 690 is separated from the micromechanical dispensing device 600 by a gap 691-691′.

In one embodiment, the gap 691-691′ is minimized to achieve substantially zero distance, providing intimate contact between the dispersion pad 690 and the dispensing device 600.

Also depicted in FIG. 6 is an optional orifice plate 695 including an orifice 696. The orifice plate 695 is arranged such that fluid dispensed by the dispensing mechanism 610 is further dispensed through the orifice 696.

Referring generally to FIG. 6, it will be understood that there are numerous fluids suitable for use with the micromechanical dispensing device 600 to control the quality or other aspects of the atmosphere for aesthetic, hygienic or mood-enhancing effects.

In one embodiment, the dispensing device 600 is arranged to dispense any of the following fluids: fragrance, perfume, therapeutic, mood-enhancing agent, pheromone, moisturizer, humectant, miticide, deodorizer, disinfectant, sanitizing agent and insecticide.

In one embodiment, any of the plurality of fluid reservoirs 620, 621, 622 contains a fluid 671, 672, 673 that comprises any of a fragrance, perfume, therapeutic, mood-enhancing agent, pheromone, moisturizer, humectant, miticide, deodorizer, disinfectant, sanitizing agent and insecticide.

In one embodiment, the micromechanical dispensing mechanism 610 comprises an electrostatically-driven membrane similar or identical to the electrostatically-driven membrane of the Kubby '865 patent.

In another embodiment, the micromechanical dispensing mechanism 610 comprises an electrostatically-actuated piston similar or identical to the electrostatically-actuated piston of the Gooray '915 patent.

In a further embodiment, the micromechanical dispensing mechanism 610 comprises a magnetically-actuated membrane similar or identical to the magnetically-actuated membrane of the Genovese patent.

In still another embodiment, the micromechanical dispensing mechanism 610 comprises a ballistic aerosol dispensing mechanism similar or identical to the ballistic aerosol dispensing mechanism of the Peeters '718 patent.

In a still further embodiment, the micromechanical dispensing mechanism 610 comprises a thermally-actuated paddle vane similar or identical to the thermally-actuated paddle-vane of the Silverbrook patent.

Referring generally to the present FIG. 6, it is believed that all information, know-how and resources needed to enable the various communications between and amongst the components depicted therein and described above in connection therewith are common and well-known to those skilled in the art.

Still referring generally to the present FIG. 6, in one embodiment, the micromechanical dispensing mechanism 610 is similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 7, there is depicted one embodiment of a micromechanical dispensing device 700 arranged to dispense a fluid into an atmosphere.

As shown, the micromechanical dispensing device 700 comprises a plurality of micromechanical dispensing mechanisms 710, 711, 712 fluidly coupled to a fluid reservoir 720.

As shown, the plurality of dispensing mechanisms 710, 711, 712 include a corresponding plurality of inlets 713, 714, 715 for receiving fluids to be dispensed by the mechanisms 710, 711, 712.

The plurality of inlets 713, 714, 715, in turn, are fluidly coupled to a channel 754.

The channel 754, in turn, is fluidly coupled to a channel port 726.

The channel port 726, in turn, is arranged to removably interconnect or mate with a corresponding fluid reservoir port 723.

The reservoir port 723, in turn, is fluidly coupled to the fluid reservoir 720.

The fluid reservoir 720, in turn, contains a fluid 771.

In one embodiment, an optional check valve 751 is interposed between the fluid reservoir 720 and the fluid reservoir port 723.

As a result of the foregoing arrangement, the fluid reservoir 720 and the fluid 771 are fluidly coupled to the plurality of dispensing mechanisms 710, 711, 712.

One skilled in the art is familiar with a variety of means to construct a removable fluid reservoir. In one embodiment, for example, the fluid reservoir 720 is similar or identical to the fluid reservoir of the Carrese patent.

As shown in FIG. 7, an included dispensing device controller 740 is arranged to actuate or control the plurality of micromechanical dispensing mechanisms 710, 711, 712 by means of suitable control signals that are communicated to the dispensing mechanisms 710, 711, 712 by means of a communication link or path 731.

In one embodiment, the device controller 740 comprises any of a number of well-known control and programming electronic circuits or devices well-known to those skilled in the art such as, for example, any of an ASIC, a PGA, a PROM, an EPROM, an EEPROM, an FPGA and a discrete circuit.

In one embodiment, the device controller 740 is comprised of electronic circuitry that is a part of the same micromechanical structure comprising the micromechanical dispensing mechanisms 710, 711, 712.

As shown in FIG. 7, in one embodiment, a program control signal 743 is communicated to the device controller 740 by means of an included controller interface 734 and a communication link or path 733.

Referring still to FIG. 7, in one embodiment, the micromechanical dispensing device 700 further comprises an optional dispensing device sensor 760. The dispensing device sensor 760, in turn, is arranged to form a dispensing device sensor signal 735 based on a concentration of an atmospheric substance 780.

Sensors responsive to the airborne concentration of substances in the atmosphere are well-known to those skilled in the art. For example, the sensor 760 may comprise a sensor similar or identical to the sensor of the Lewis patent.

In one embodiment, the atmospheric substance 780 comprises the fluid 771 that is dispensed by the dispensing device 700.

As shown, in one embodiment, the dispensing device sensor 760 is arranged to communicate the dispensing device sensor signal 735 to the controller 740 by means of a communication link or path 732. In turn, the device controller 740 is arranged to actuate or control the dispensing mechanisms 710, 711, 711 based at least in part on the dispensing device sensor signal 735.

In another embodiment, the dispensing device sensor signal 735 is communicated to an included dispensing device sensor interface 762 by means of a communication link or path 761.

Still referring to FIG. 7, in one embodiment, the micromechanical dispensing device 700 comprises a dispersion pad 790 positioned to receive a fluid that is dispensed by the micromechanical dispensing mechanisms 710, 711, 712.

In one embodiment, the dispersion pad 790 comprises any natural or synthetic material or polymer, fiber or strand, either singular or woven, twisted, braided, bundled, molded or shaped in a manner that transports fluid or vapors by capillary action or that can serve as a support medium for the fluid or vapors.

The dispersion pad 790 is separated from the micromechanical dispensing device 700 by a gap 791-791′.

In one embodiment, the gap 791-791′ is minimized to achieve substantially zero distance, providing intimate contact between the dispersion pad 790 and the dispensing device 700.

Also depicted in FIG. 7 is an optional orifice plate 795 including an orifice 796. The orifice plate 795 is arranged such that fluid dispensed by the dispensing mechanisms 710, 711, 712 is further dispensed through the orifice 796.

Referring generally to FIG. 7, it will be understood that there are numerous fluids suitable for use with the micromechanical dispensing device 700 to control the quality or other aspects of the atmosphere for aesthetic, hygienic or mood-enhancing effects.

In one embodiment, the dispensing device 700 is arranged to dispense any of the following fluids: fragrance, perfume, therapeutic, mood-enhancing agent, pheromone, moisturizer, humectant, miticide, deodorizer, disinfectant, sanitizing agent and insecticide.

In one embodiment, the fluid reservoir 720 contains a fluid 771 that comprises any of a fragrance, perfume, therapeutic, mood-enhancing agent, pheromone, moisturizer, humectant, miticide, deodorizer, disinfectant, sanitizing agent and insecticide.

In one embodiment, any of the plurality of micromechanical dispensing mechanisms 710, 711, 712 comprise an electrostatically-driven membrane similar or identical to the electrostatically-driven membrane of the Kubby '865 patent.

In another embodiment, any of the plurality of micromechanical dispensing mechanisms 710, 711, 712 comprise an electrostatically-actuated piston similar or identical to the electrostatically-actuated piston of the Gooray '915 patent.

In a further embodiment, any of the plurality of micromechanical dispensing mechanisms 710, 711, 712 comprise a magnetically-actuated membrane similar or identical to the magnetically-actuated membrane of the Genovese patent.

In still another embodiment, any of the plurality of micromechanical dispensing mechanisms 710, 711, 712 comprise a ballistic aerosol dispensing mechanism similar or identical to the ballistic aerosol dispensing mechanism of the Peeters '718 patent.

In a still further embodiment, any of the plurality of micromechanical dispensing mechanisms 710, 711, 712 comprise a thermally-actuated paddle vane similar or identical to the thermally-actuated paddle-vane of the Silverbrook patent.

Referring generally to the present FIG. 7, it is believed that all information, know-how and resources needed to enable the various communications between and amongst the components depicted therein and described above in connection therewith are common and well-known to those skilled in the art.

Still referring generally to the present FIG. 7, in one embodiment, any of the plurality of micromechanical dispensing mechanisms 710, 711, 712 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

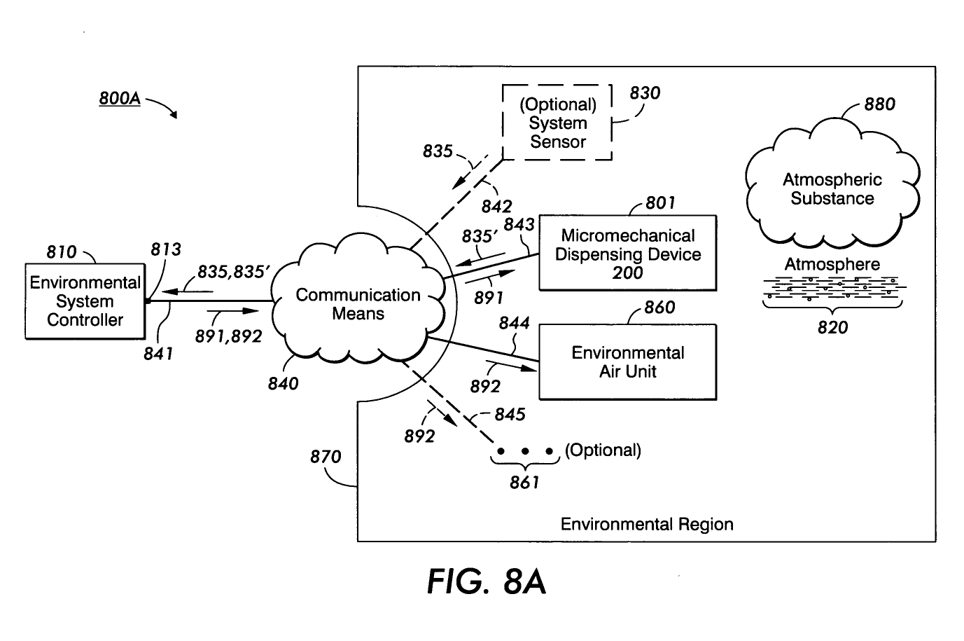

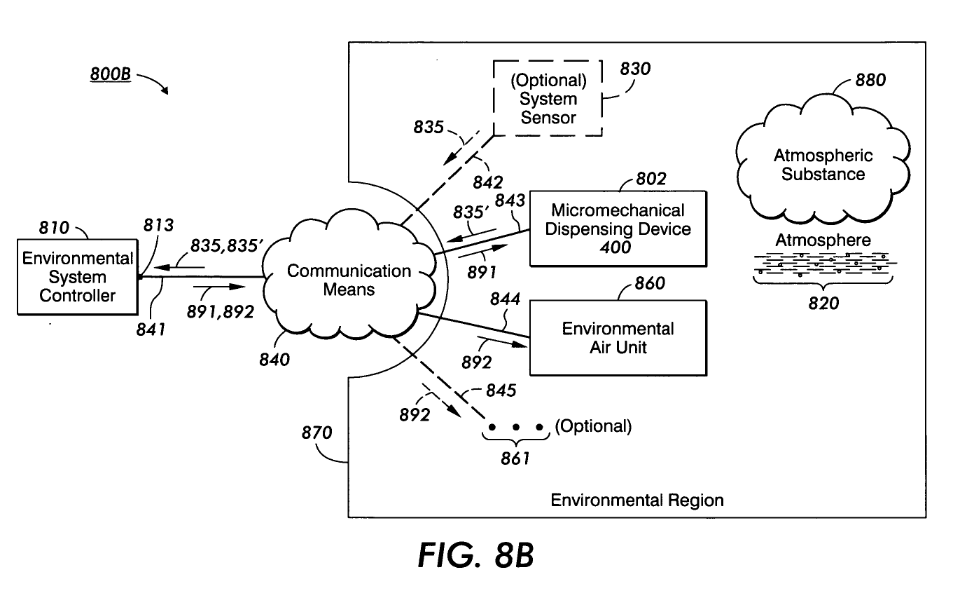

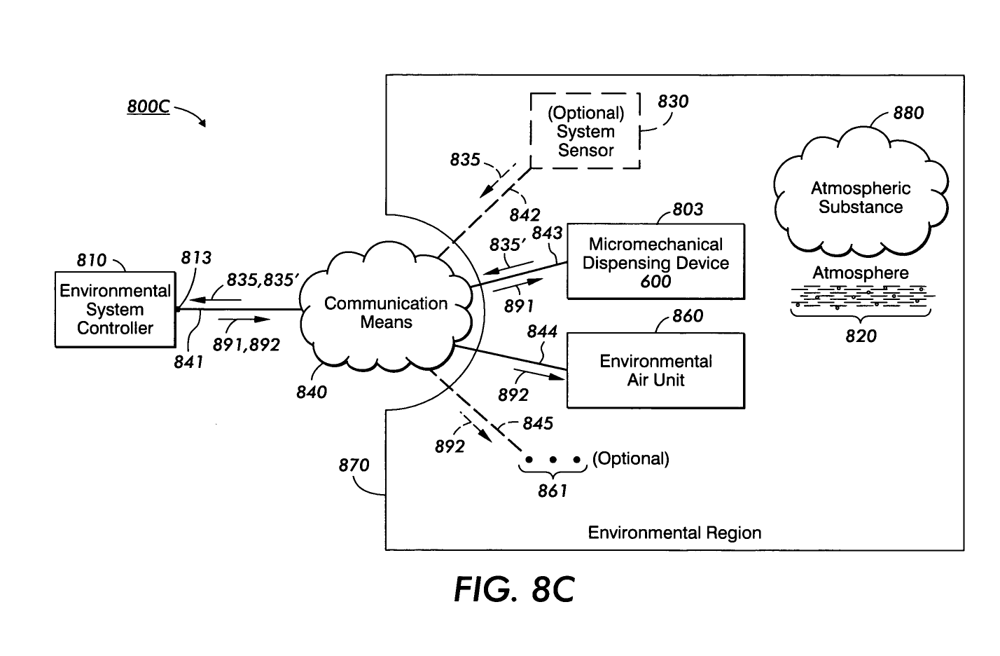

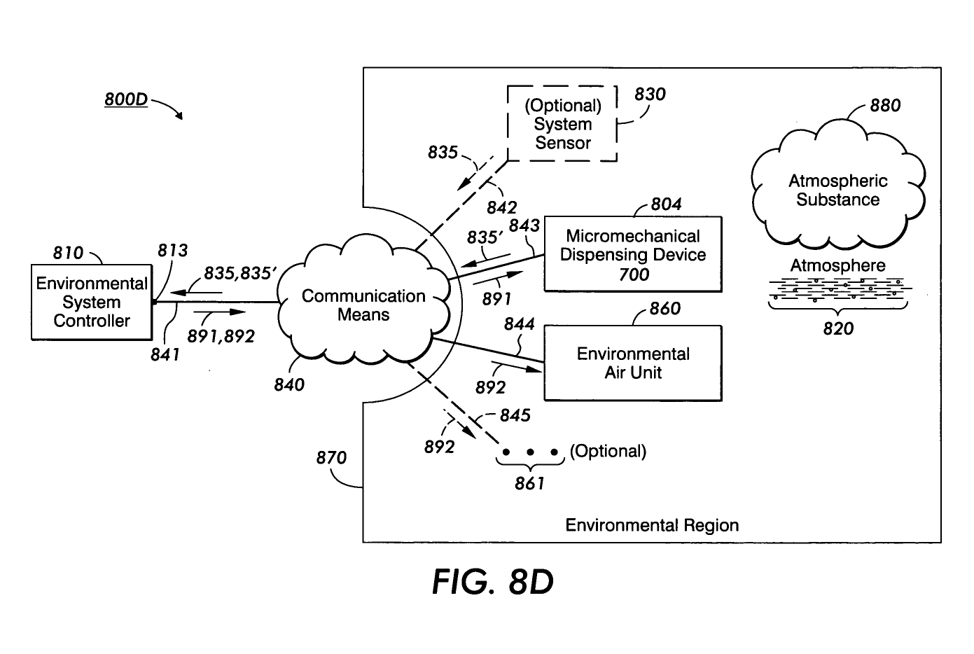

Referring now to FIG. 8A, there is depicted an environmental system 800A including a micromechanical dispensing device 801. The micromechanical dispensing device 801, in turn, comprises the micromechanical dispensing device 200 described in connection with FIG. 2 above.

As shown in FIG. 8A, the environmental system 800A comprises an environmental system controller 810. The environmental system controller 810, in turn, comprises a controller communication interface 813. The environmental system controller 810 is arranged to communicate with one or more environmental air units 860, 861 by means of the controller communication interface 813, a communication link or path 841, a communication means 840, a communication link or path 844 and an optional communication link or path 845.

As shown, the one or more environmental air units 860, 861 are located in an environmental region 870.

As shown, the environmental system controller 810 is arranged to actuate or control the one or more environmental air units 860, 861 by means of suitable control signals 892 that are communicated to the one or more environmental air units 860, 861 by means of the controller communication interface 813, the link or path 841, the communication means 840 and the links or paths 844, 845.

As shown, the environmental region 870 comprises an atmosphere 820. Moreover, the one or more environmental air units 860, 861 are arranged to alter or control one or more physical properties of the atmosphere 820.

In one embodiment, any of the one or more environmental air units 860, 861 are arranged to alter or control any of the following included physical properties of the atmosphere 820: temperature, humidity, circulation and cleanliness.

In one embodiment, any of the one or more environmental air units 860, 861 comprise an air heating device such as, for example, any of a furnace, an electric heater and a heat pump.

In one embodiment, any of the one or more environmental air units 860, 861 comprise an air cooling device such as, for example any of an air conditioner and a heat pump.

In one embodiment, any of the one or more environmental air units 860, 861 comprise an air humidity control device including, without limitation, means to increase the humidity, decrease the humidity, or both. In one embodiment, for example, any of the environmental air units 860, 861 comprises a device similar or identical to the humidifier described in the Bloemer patent.

In one embodiment, any of the one or more environmental air units 860, 861 comprise an air circulating or flow device such as, for example, any of a blower, a fan and a damper.

In one embodiment, any of the one or more environmental air units 860, 861 comprise an air cleaning device such as, for example, any of a filter, a purifier, an ozone generator and an electrostatic precipitator.

Still referring to FIG. 8A, the environmental system controller 810 is further arranged to communicate with the micromechanical dispensing device 801. The micromechanical dispensing device 801 is located in the environmental region 870.

As shown, the environmental system controller 810 is arranged to actuate or control the micromechanical dispensing device 801 by means of suitable control signals 891 that are communicated to the micromechanical dispensing device 801 by means of the controller communication interface 813, the link or path 841, the communication means 840 and a communication link or path 843.

With momentary cross-reference to FIG. 2, as mentioned above, the micromechanical dispensing device 801 in the present FIG. 8A comprises the micromechanical dispensing device 200. Hence, the aforementioned control signals 891 in the present FIG. 8A correspond to the program control signal 243 in FIG. 2. As described in connection with FIG. 2 above, the program control signal 243 is communicated to the device controller 240 comprised in the dispensing device 200 by means of the included controller interface 234 and the communication link or path 233.

With continued cross-reference to FIG. 2, the micromechanical dispensing device 801 in the present FIG. 8A, corresponding to the dispensing device 200, comprises one or more micromechanical dispensing mechanisms 210, 212, each of the one or more micromechanical dispensing mechanisms 210, 212 being arranged to fluidly couple by means of channels 254, 255 to a corresponding fluid reservoir of one or more fluid reservoirs 220, 222. The one or more fluid reservoirs 220, 222, in turn, contain a corresponding one or more fluids 271, 273. As a result, the micromechanical dispensing device 801 in the present FIG. 8A is arranged to dispense the one or more fluids 271, 273 into the atmosphere 820.

With continued cross-reference to FIG. 2, in one embodiment, any of the one or more fluid reservoirs 220, 222 contain a fluid 271, 273 comprising any of a fragrance, perfume, therapeutic, mood-enhancing agent, pheromone, moisturizer, humectant, miticide, deodorizer, disinfectant, sanitizing agent and insecticide.

As described in connection with FIG. 2 above, in one embodiment, any of the one or more micromechanical dispensing mechanisms 210, 212 comprised in the micromechanical dispensing device 801 in the present FIG. 8A comprise any of an electrostatically-driven membrane, an electrostatically-actuated piston, a magnetically-actuated membrane, a thermally-actuated paddle vane and a ballistic aerosol dispensing mechanism.

Referring again to the present FIG. 8A, in one embodiment the environmental system 800A further comprises an optional system sensor 830 that is located in the environmental region 870. For example, the system sensor 830 may be similar or identical to the sensor of the Lewis patent.

As shown, the system sensor 830 is arranged to form a system sensor signal 835 based on an atmospheric substance 880 comprised in the atmosphere 820. The system sensor 830 is further arranged to communicate the system sensor signal 835 to the environmental system controller 810 by means of a communication link or path 842, the communication means 840, and the link or path 841.

In one embodiment, the environmental system controller 810 is arranged to actuate or control any of the one or more environmental air units 860, 861 based at least in part on the system sensor signal 835.

In one embodiment, the environmental system controller 810 is arranged to actuate or control the micromechanical dispensing device 801 based at least in part on the system sensor signal 835.

In one embodiment, the atmospheric substance 880 comprises any of the one or more fluids 271, 273 that are dispensed by the micromechanical dispensing device 801.

In one embodiment, the atmospheric substance 880 comprises any of a human body fluid in liquid or gaseous form and an odor or fragrance that is formed by a human body.

For example, in one embodiment the atmospheric substance 880 comprises an odor or fragrance based on an environmental discomfort that is being experienced by one or more humans located in the environmental region 870 as a result of an environmental problem such as, for example, excessive heat, excessive cold, excessive humidity, excessive dryness, the air containing an unpleasant odor, etc. For example, the odor or fragrance might comprise human perspiration or human “body odor” as a result of excessive heat or excessive humidity.

With cross-reference to FIG. 2, in one embodiment the dispensing device 801 in FIG. 8A (which corresponds to the dispensing device 200 in FIG. 2) further comprises the optional dispensing device sensor 260 that is described in connection with FIG. 2. As shown in the present FIG. 8A, in this embodiment the dispensing device sensor 260 of the dispensing device 801 is arranged to form a system sensor signal 835′ (corresponding to the dispensing device sensor signal 235 as communicated to the dispensing device sensor interface 262 in FIG. 2) based on the atmospheric substance 880 comprised in the atmosphere 820 and to communicate the system sensor signal 835′ to the environmental system controller 810 by means of the link or path 843, the communication means 840 and the link or path 841.

Still referring to FIG. 8A, in one embodiment, the environmental system controller 810 is arranged to actuate or control any of the one or more environmental air units 860, 861 based at least in part on the system sensor signal 835′.

In one embodiment, the environmental system controller 810 is arranged to actuate or control the micromechanical dispensing device 801 based at least in part on the system sensor signal 835′.

In one embodiment, the communication means 840 and the communication links or paths 841, 842, 843, 844 and 845 comprise a communication network.

In one embodiment, the communication means 840 and the links or paths 841, 842, 843, 844 and 845 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 8A, it is believed that all information, know-how and resources needed to enable the various communications between and amongst the components depicted therein and described above in connection therewith are common and well-known to those skilled in the art.

Still referring generally to the present FIG. 8A, in one embodiment, any of the one or more micromechanical dispensing mechanisms 210, 212 comprised in the micromechanical dispensing device 801 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 8B, there is depicted an environmental system 800B including a micromechanical dispensing device 802. The micromechanical dispensing device 802, in turn, comprises the micromechanical dispensing device 400 described in connection with FIG. 4 above.

As shown in FIG. 8B, the environmental system 800B comprises an environmental system controller 810. The environmental system controller 810, in turn, comprises a controller communication interface 813. The environmental system controller 810 is arranged to communicate with one or more environmental air units 860, 861 by means of the controller communication interface 813, a communication link or path 841, a communication means 840, a communication link or path 844 and an optional communication link or path 845.

As shown, the one or more environmental air units 860, 861 are located in an environmental region 870.

As shown, the environmental system controller 810 is arranged to actuate or control the one or more environmental air units 860, 861 by means of suitable control signals 892 that are communicated to the one or more environmental air units 860, 861 by means of the controller communication interface 813, the link or path 841, the communication means 840 and the links or paths 844, 845.

As shown, the environmental region 870 comprises an atmosphere 820. Moreover, the one or more environmental air units 860, 861 are arranged to alter or control one or more physical properties of the atmosphere 820.

In one embodiment, any of the one or more environmental air units 860, 861 are arranged to alter or control any of the following included physical properties of the atmosphere 820: temperature, humidity, circulation and cleanliness.

In one embodiment, any of the one or more environmental air units 860, 861 comprise an air heating device such as, for example, any of a furnace, an electric heater and a heat pump.

In one embodiment, any of the one or more environmental air units 860, 861 comprise an air cooling device such as, for example any of an air conditioner and a heat pump.

In one embodiment, any of the one or more environmental air units 860, 861 comprise an air humidity control device including, without limitation, means to increase the humidity, decrease the humidity, or both. In one embodiment, for example, any of the environmental air units 860, 861 comprises a device similar or identical to the humidifier described in the Bloemer patent.

In one embodiment, any of the one or more environmental air units 860, 861 comprise an air circulating or flow device such as, for example, any of a blower, a fan and a damper.

In one embodiment, any of the one or more environmental air units 860, 861 comprise an air cleaning device such as, for example, any of a filter, a purifier, an ozone generator and an electrostatic precipitator.

Still referring to FIG. 8B, the environmental system controller 810 is further arranged to communicate with the micromechanical dispensing device 802. The micromechanical dispensing device 802 is located in the environmental region 870.

As shown, the environmental system controller 810 is arranged to actuate or control the micromechanical dispensing device 802 by means of suitable control signals 891 that are communicated to the micromechanical dispensing device 802 by means of the controller communication interface 813, the link or path 841, the communication means 840 and a communication link or path 843.

With momentary cross-reference to FIG. 4, as mentioned above, the micromechanical dispensing device 802 in the present FIG. 8B comprises the micromechanical dispensing device 400. Hence, the aforementioned control signals 891 in the present FIG. 8B correspond to the program control signal 443 in FIG. 4. As described in connection with FIG. 4 above, the program control signal 443 is communicated to the device controller 440 comprised in the dispensing device 400 by means of the included controller interface 434 and the communication link or path 433.

With continued cross-reference to FIG. 4, the micromechanical dispensing device 802 in the present FIG. 8B, corresponding to the dispensing device 400, comprises a plurality of micromechanical dispensing mechanisms 410, 411, 412, each of the plurality of micromechanical dispensing mechanisms 410, 411, 412 being arranged to fluidly couple by means of channels 454, 455, 456 to a corresponding fluid reservoir of a plurality of fluid reservoirs 420, 421, 422. The plurality of fluid reservoirs 420, 421, 422, in turn, contain a corresponding plurality of fluids 471, 472, 473. As a result, the micromechanical dispensing device 802 in the present FIG. 8B is arranged to dispense the plurality of fluids 471, 472, 473 into the atmosphere 820.

With continued cross-reference to FIG. 4, in one embodiment, any of the plurality of fluid reservoirs 420, 421, 422 contain a fluid 471, 472, 473 comprising any of a fragrance, perfume, therapeutic, mood-enhancing agent, pheromone, moisturizer, humectant, miticide, deodorizer, disinfectant, sanitizing agent and insecticide.

As described in connection with FIG. 4 above, in one embodiment, any of the plurality of micromechanical dispensing mechanisms 410, 411, 412 comprised in the micromechanical dispensing device 802 in the present FIG. 8B comprise any of an electrostatically-driven membrane, an electrostatically-actuated piston, a magnetically-actuated membrane, a thermally-actuated paddle vane and a ballistic aerosol dispensing mechanism.

Referring again to the present FIG. 8B, in one embodiment the environmental system 800B further comprises an optional system sensor 830 that is located in the environmental region 870. For example, the system sensor 830 may be similar or identical to the sensor of the Lewis patent.

As shown, the system sensor 830 is arranged to form a system sensor signal 835 based on an atmospheric substance 880 comprised in the atmosphere 820. The system sensor 830 is further arranged to communicate the system sensor signal 835 to the environmental system controller 810 by means of a communication link or path 842, the communication means 840, and the link or path 841.

In one embodiment, the environmental system controller 810 is arranged to actuate or control any of the one or more environmental air units 860, 861 based at least in part on the system sensor signal 835.

In one embodiment, the environmental system controller 810 is arranged to actuate or control the micromechanical dispensing device 802 based at least in part on the system sensor signal 835.

In one embodiment, the atmospheric substance 880 comprises any of the plurality of fluids 471, 472, 473 that are dispensed by the micromechanical dispensing device 802.

In one embodiment, the atmospheric substance 880 comprises any of a human body fluid in liquid or gaseous form and an odor or fragrance that is formed by a human body.

For example, in one embodiment the atmospheric substance 880 comprises an odor or fragrance based on an environmental discomfort that is being experienced by one or more humans located in the environmental region 870 as a result of an environmental problem such as, for example, excessive heat, excessive cold, excessive humidity, excessive dryness, the air containing an unpleasant odor, etc. For example, the odor or fragrance might comprise human perspiration or human “body odor” as a result of excessive heat or excessive humidity.

With cross-reference to FIG. 4, in one embodiment the dispensing device 802 in FIG. 8B (which corresponds to the dispensing device 400 in FIG. 4) further comprises the optional dispensing device sensor 460 that is described in connection with FIG. 4. As shown in the present FIG. 8B, in this embodiment the dispensing device sensor 460 of the dispensing device 802 is arranged to form a system sensor signal 835′ (corresponding to the dispensing device sensor signal 435 as communicated to the dispensing device sensor interface 462 in FIG. 4) based on the atmospheric substance 880 comprised in the atmosphere 820 and to communicate the system sensor signal 835′ to the environmental system controller 810 by means of the link or path 843, the communication means 840 and the link or path 841.

Still referring to FIG. 8B, in one embodiment, the environmental system controller 810 is arranged to actuate or control any of the one or more environmental air units 860, 861 based at least in part on the system sensor signal 835′.

In one embodiment, the environmental system controller 810 is arranged to actuate or control the micromechanical dispensing device 802 based at least in part on the system sensor signal 835′.

In one embodiment, the communication means 840 and the communication links or paths 841, 842, 843, 844 and 845 comprise a communication network.

In one embodiment, the communication means 840 and the links or paths 841, 842, 843, 844 and 845 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 8B, it is believed that all information, know-how and resources needed to enable the various communications between and amongst the components depicted therein and described above in connection therewith are common and well-known to those skilled in the art.

Still referring generally to the present FIG. 8B, in one embodiment, any of the plurality of micromechanical dispensing mechanisms 410, 411, 412 comprised in the micromechanical dispensing device 802 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 8C, there is depicted an environmental system 800C including a micromechanical dispensing device 803. The micromechanical dispensing device 803, in turn, comprises the micromechanical dispensing device 600 described in connection with FIG. 6 above.

As shown in FIG. 8C, the environmental system 800C comprises an environmental system controller 810. The environmental system controller 810, in turn, comprises a controller communication interface 813. The environmental system controller 810 is arranged to communicate with one or more environmental air units 860, 861 by means of the controller communication interface 813, a communication link or path 841, a communication means 840, a communication link or path 844 and an optional communication link or path 845.

As shown, the one or more environmental air units 860, 861 are located in an environmental region 870.

As shown, the environmental system controller 810 is arranged to actuate or control the one or more environmental air units 860, 861 by means of suitable control signals 892 that are communicated to the one or more environmental air units 860, 861 by means of the controller communication interface 813, the link or path 841, the communication means 840 and the links or paths 844, 845.

As shown, the environmental region 870 comprises an atmosphere 820. Moreover, the one or more environmental air units 860, 861 are arranged to alter or control one or more physical properties of the atmosphere 820.

In one embodiment, any of the one or more environmental air units 860, 861 are arranged to alter or control any of the following included physical properties of the atmosphere 820: temperature, humidity, circulation and cleanliness.

In one embodiment, any of the one or more environmental air units 860, 861 comprise an air heating device such as, for example, any of a furnace, an electric heater and a heat pump.

In one embodiment, any of the one or more environmental air units 860, 861 comprise an air cooling device such as, for example any of an air conditioner and a heat pump.

In one embodiment, any of the one or more environmental air units 860, 861 comprise an air humidity control device including, without limitation, means to increase the humidity, decrease the humidity, or both. In one embodiment, for example, any of the environmental air units 860, 861 comprises a device similar or identical to the humidifier described in the Bloemer patent.

In one embodiment, any of the one or more environmental air units 860, 861 comprise an air circulating or flow device such as, for example, any of a blower, a fan and a damper.

In one embodiment, any of the one or more environmental air units 860, 861 comprise an air cleaning device such as, for example, any of a filter, a purifier, an ozone generator and an electrostatic precipitator.

Still referring to FIG. 8C, the environmental system controller 810 is further arranged to communicate with the micromechanical dispensing device 803. The micromechanical dispensing device 803 is located in the environmental region 870.

As shown, the environmental system controller 810 is arranged to actuate or control the micromechanical dispensing device 803 by means of suitable control signals 891 that are communicated to the micromechanical dispensing device 803 by means of the controller communication interface 813, the link or path 841, the communication means 840 and a communication link or path 843.

With momentary cross-reference to FIG. 6, as mentioned above, the micromechanical dispensing device 803 in the present FIG. 8C comprises the micromechanical dispensing device 600. Hence, the aforementioned control signals 891 in the present FIG. 8C correspond to the program control signal 643 in FIG. 6. As described in connection with FIG. 6 above, the program control signal 643 is communicated to the device controller 640 comprised in the dispensing device 600 by means of the included controller interface 634 and the communication link or path 633.

With continued cross-reference to FIG. 6, the micromechanical dispensing device 803 in the present FIG. 8C, corresponding to the dispensing device 600, comprises a micromechanical dispensing mechanism 610 that is fluidly coupled to a valve 665 by means of channel 611-611′. As described in connection with FIG. 6 above, the valve 665, in turn, is arranged to selectively fluidly couple the micromechanical dispensing mechanism 610 to a plurality of fluid reservoirs 620, 621, 622 by means of channels 611-611′ and 612. The plurality of fluid reservoirs 620, 621, 622, in turn, contain a corresponding plurality of fluids 671, 672, 673. As a result, the micromechanical dispensing device 803 in the present FIG. 8C is arranged to dispense the plurality of fluids 671, 672, 673 into the atmosphere 820.

With continued cross-reference to FIG. 6, in one embodiment, any of the plurality of fluid reservoirs 620, 621, 622 contain a fluid 671, 672, 673 comprising any of a fragrance, perfume, therapeutic, mood-enhancing agent, pheromone, moisturizer, humectant, miticide, deodorizer, disinfectant, sanitizing agent and insecticide.

As described in connection with FIG. 6 above, in one embodiment, the micromechanical dispensing mechanism 610 comprised in the micromechanical dispensing device 803 in the present FIG. 8C comprise any of an electrostatically-driven membrane, an electrostatically-actuated piston, a magnetically-actuated membrane, a thermally-actuated paddle vane and a ballistic aerosol dispensing mechanism.

Referring again to the present FIG. 8C, in one embodiment the environmental system 800C further comprises an optional system sensor 830 that is located in the environmental region 870. For example, the system sensor 830 may be similar or identical to the sensor of the Lewis patent.

As shown, the system sensor 830 is arranged to form a system sensor signal 835 based on an atmospheric substance 880 comprised in the atmosphere 820. The system sensor 830 is further arranged to communicate the system sensor signal 835 to the environmental system controller 810 by means of a communication link or path 842, the communication means 840, and the link or path 841.

In one embodiment, the environmental system controller 810 is arranged to actuate or control any of the one or more environmental air units 860, 861 based at least in part on the system sensor signal 835.

In one embodiment, the environmental system controller 810 is arranged to actuate or control the micromechanical dispensing device 803 based at least in part on the system sensor signal 835.

In one embodiment, the atmospheric substance 880 comprises any of the plurality of fluids 671, 672, 673 that are dispensed by the micromechanical dispensing device 803.

In one embodiment, the atmospheric substance 880 comprises any of a human body fluid in liquid or gaseous form and an odor or fragrance that is formed by a human body.

For example, in one embodiment the atmospheric substance 880 comprises an odor or fragrance based on an environmental discomfort that is being experienced by one or more humans located in the environmental region 870 as a result of an environmental problem such as, for example, excessive heat, excessive cold, excessive humidity, excessive dryness, the air containing an unpleasant odor, etc. For example, the odor or fragrance might comprise human perspiration or human “body odor” as a result of excessive heat or excessive humidity.

With cross-reference to FIG. 6, in one embodiment the dispensing device 803 in FIG. 8C (which corresponds to the dispensing device 600 in FIG. 6) further comprises the optional dispensing device sensor 660 that is described in connection with FIG. 6. As shown in the present FIG. 8C, in this embodiment the dispensing device sensor 660 of the dispensing device 803 is arranged to form a system sensor signal 835′ (corresponding to the dispensing device sensor signal 635 as communicated to the dispensing device sensor interface 662 in FIG. 6) based on the atmospheric substance 880 comprised in the atmosphere 820 and to communicate the system sensor signal 835′ to the environmental system controller 810 by means of the link or path 843, the communication means 840 and the link or path 841.

Still referring to FIG. 8C, in one embodiment, the environmental system controller 810 is arranged to actuate or control any of the one or more environmental air units 860, 861 based at least in part on the system sensor signal 835′.

In one embodiment, the environmental system controller 810 is arranged to actuate or control the micromechanical dispensing device 803 based at least in part on the system sensor signal 835′.

In one embodiment, the communication means 840 and the communication links or paths 841, 842, 843, 844 and 845 comprise a communication network.

In one embodiment, the communication means 840 and the links or paths 841, 842, 843, 844 and 845 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 8C, it is believed that all information, know-how and resources needed to enable the various communications between and amongst the components depicted therein and described above in connection therewith are common and well-known to those skilled in the art.

Still referring generally to the present FIG. 8C, in one embodiment, any of the plurality of micromechanical dispensing mechanisms 610, 611, 612 comprised in the micromechanical dispensing device 803 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

Referring now to FIG. 8D, there is depicted an environmental system 800D including a micromechanical dispensing device 804. The micromechanical dispensing device 804, in turn, comprises the micromechanical dispensing device 700 described in connection with FIG. 7 above.

As shown in FIG. 8D, the environmental system 800D comprises an environmental system controller 810. The environmental system controller 810, in turn, comprises a controller communication interface 813. The environmental system controller 810 is arranged to communicate with one or more environmental air units 860, 861 by means of the controller communication interface 813, a communication link or path 841, a communication means 840, a communication link or path 844 and an optional communication link or path 845.

As shown, the one or more environmental air units 860, 861 are located in an environmental region 870.

As shown, the environmental system controller 810 is arranged to actuate or control the one or more environmental air units 860, 861 by means of suitable control signals 892 that are communicated to the one or more environmental air units 860, 861 by means of the controller communication interface 813, the link or path 841, the communication means 840 and the links or paths 844, 845.

As shown, the environmental region 870 comprises an atmosphere 820. Moreover, the one or more environmental air units 860, 861 are arranged to alter or control one or more physical properties of the atmosphere 820.

In one embodiment, any of the one or more environmental air units 860, 861 are arranged to alter or control any of the following included physical properties of the atmosphere 820: temperature, humidity, circulation and cleanliness.

In one embodiment, any of the one or more environmental air units 860, 861 comprise an air heating device such as, for example, any of a furnace, an electric heater and a heat pump.

In one embodiment, any of the one or more environmental air units 860, 861 comprise an air cooling device such as, for example any of an air conditioner and a heat pump.

In one embodiment, any of the one or more environmental air units 860, 861 comprise an air humidity control device including, without limitation, means to increase the humidity, decrease the humidity, or both. In one embodiment, for example, any of the environmental air units 860, 861 comprises a device similar or identical to the humidifier described in the Bloemer patent.

In one embodiment, any of the one or more environmental air units 860, 861 comprise an air circulating or flow device such as, for example, any of a blower, a fan and a damper.

In one embodiment, any of the one or more environmental air units 860, 861 comprise an air cleaning device such as, for example, any of a filter, a purifier, an ozone generator and an electrostatic precipitator.

Still referring to FIG. 8D, the environmental system controller 810 is further arranged to communicate with the micromechanical dispensing device 804. The micromechanical dispensing device 804 is located in the environmental region 870.

As shown, the environmental system controller 810 is arranged to actuate or control the micromechanical dispensing device 804 by means of suitable control signals 891 that are communicated to the micromechanical dispensing device 804 by means of the controller communication interface 813, the link or path 841, the communication means 840 and a communication link or path 843.

With momentary cross-reference to FIG. 7, as mentioned above, the micromechanical dispensing device 804 in the present FIG. 8D comprises the micromechanical dispensing device 700. Hence, the aforementioned control signals 891 in the present FIG. 8D correspond to the program control signal 743 in FIG. 7. As described in connection with FIG. 7 above, the program control signal 743 is communicated to the device controller 740 comprised in the dispensing device 700 by means of the included controller interface 734 and the communication link or path 733.

With continued cross-reference to FIG. 7, the micromechanical dispensing device 804 in the present FIG. 8D, corresponding to the dispensing device 700, comprises a plurality of micromechanical dispensing mechanisms 710, 711, 712 that are arranged to fluidly couple by means of channel 754 to a fluid reservoir 720. The fluid reservoir 720, in turn, contains a corresponding fluid 771. As a result, the micromechanical dispensing device 804 in the present FIG. 8D is arranged to dispense the fluid 771 into the atmosphere 820.

With continued cross-reference to FIG. 7, in one embodiment, the fluid reservoir 720 contains a fluid 771 comprising any of a fragrance, perfume, therapeutic, mood-enhancing agent, pheromone, moisturizer, humectant, miticide, deodorizer, disinfectant, sanitizing agent and insecticide.

As described in connection with FIG. 7 above, in one embodiment, any of the plurality of micromechanical dispensing mechanisms 710, 711, 712 comprised in the micromechanical dispensing device 804 in the present FIG. 8D comprise any of an electrostatically-driven membrane, an electrostatically-actuated piston, a magnetically-actuated membrane, a thermally-actuated paddle vane and a ballistic aerosol dispensing mechanism.

Referring again to the present FIG. 8D, in one embodiment the environmental system 800D further comprises an optional system sensor 830 that is located in the environmental region 870. For example, the system sensor 830 may be similar or identical to the sensor of the Lewis patent.

As shown, the system sensor 830 is arranged to form a system sensor signal 835 based on an atmospheric substance 880 comprised in the atmosphere 820. The system sensor 830 is further arranged to communicate the system sensor signal 835 to the environmental system controller 810 by means of a communication link or path 842, the communication means 840, and the link or path 841.

In one embodiment, the environmental system controller 810 is arranged to actuate or control any of the one or more environmental air units 860, 861 based at least in part on the system sensor signal 835.

In one embodiment, the environmental system controller 810 is arranged to actuate or control the micromechanical dispensing device 804 based at least in part on the system sensor signal 835.

In one embodiment, the atmospheric substance 880 comprises the fluid 771 that is dispensed by the micromechanical dispensing device 804.

In one embodiment, the atmospheric substance 880 comprises any of a human body fluid in liquid or gaseous form and an odor or fragrance that is formed by a human body.

For example, in one embodiment the atmospheric substance 880 comprises an odor or fragrance based on an environmental discomfort that is being experienced by one or more humans located in the environmental region 870 as a result of an environmental problem such as, for example, excessive heat, excessive cold, excessive humidity, excessive dryness, the air containing an unpleasant odor, etc. For example, the odor or fragrance might comprise human perspiration or human “body odor” as a result of excessive heat or excessive humidity.

With cross-reference to FIG. 7, in one embodiment the dispensing device 804 in FIG. 8D (which corresponds to the dispensing device 700 in FIG. 7) further comprises the optional dispensing device sensor 760 that is described in connection with FIG. 7. As shown in the present FIG. 8D, in this embodiment the dispensing device sensor 760 of the dispensing device 804 is arranged to form a system sensor signal 835′ (corresponding to the dispensing device sensor signal 735 as communicated to the dispensing device sensor interface 762 in FIG. 7) based on the atmospheric substance 880 comprised in the atmosphere 820 and to communicate the system sensor signal 835′ to the environmental system controller 810 by means of the link or path 843, the communication means 840 and the link or path 841.

Still referring to FIG. 8D, in one embodiment, the environmental system controller 810 is arranged to actuate or control any of the one or more environmental air units 860, 861 based at least in part on the system sensor signal 835′.

In one embodiment, the environmental system controller 810 is arranged to actuate or control the micromechanical dispensing device 804 based at least in part on the system sensor signal 835′.

In one embodiment, the communication means 840 and the communication links or paths 841, 842, 843, 844 and 845 comprise a communication network.

In one embodiment, the communication means 840 and the links or paths 841, 842, 843, 844 and 845 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring generally to the present FIG. 8D, it is believed that all information, know-how and resources needed to enable the various communications between and amongst the components depicted therein and described above in connection therewith are common and well-known to those skilled in the art.

Still referring generally to the present FIG. 8D, in one embodiment, any of the plurality of micromechanical dispensing mechanisms 710, 711, 712 comprised in the micromechanical dispensing device 804 are similar or identical to any of the micromechanical or micro-electromechanical fluid ejectors described in the following patents: Coleman '198, Coleman '841, Genovese, Gooray '015, Gooray '915, Gooray '130, Gooray '311, Gooray '169, Gooray '335, Kubby '865, Kubby '448, Peeters '718 and Silverbrook.

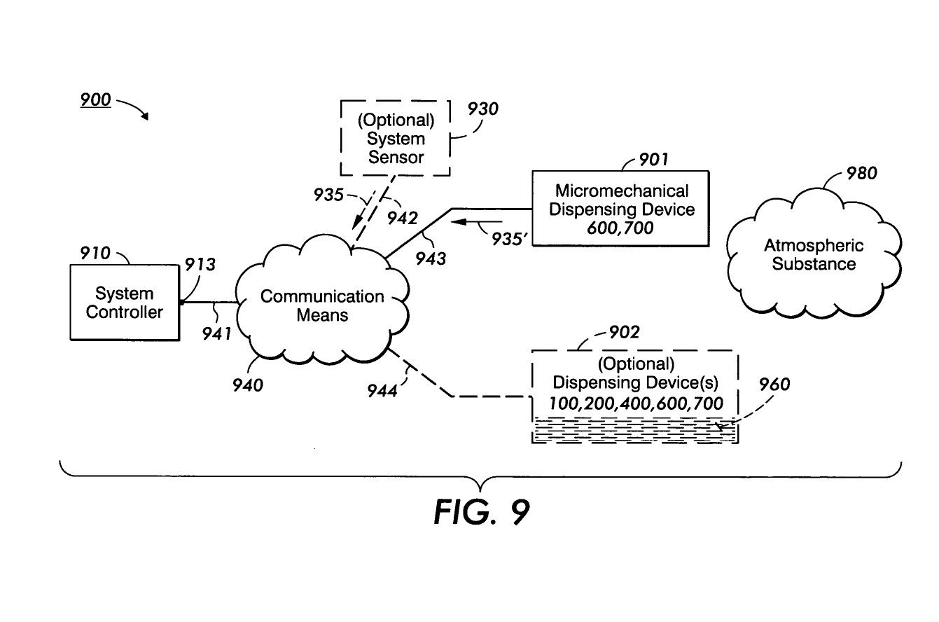

Referring now to FIG. 9, there is depicted a dispensing system 900 for dispensing one or more fluids into an atmosphere. As shown, the dispensing system 900 comprises a dispensing system controller 910, a communication means 940 and a micromechanical dispensing device 901.

In one embodiment the dispensing system 900 comprises only a single dispensing device, namely, the dispensing device 901.

In another embodiment, the dispensing system 900 comprises the dispensing device 901 and at least one additional dispensing device 902. The at least one additional dispensing device 902, in turn, comprises any of the dispensing devices 100, 200, 400, 600 and 700 that are respectively described in connection with FIGS. 1, 2, 4, 6 and 7. As shown, the optional at least one additional dispensing device is arranged to dispense one or more fluids 960.

As shown, the dispensing system controller 910 actuates or controls the dispensing device 901 by means of suitable control signals that are communicated to the dispensing device 901 by means of an included controller communication interface 913, a communication link or path 941, the communication means 940 and a communication link or path 943. The system controller 910 further actuates or controls the optional at least one additional dispensing device 902 by means of suitable control signals that are communicated to the dispensing device 902 by means of the controller communication interface 913, the link or path 941, the communication means 940 and a communication link or path 944.

In one embodiment, an optional system sensor 930 is provided. For example, the system sensor 930 may be similar or identical to the sensor of the Lewis patent.

As shown, the system sensor 930 forms a system sensor signal 935 based on a concentration of an atmospheric substance 980 and communicates the system sensor signal 935 to the system controller 910 by means of a communication link or path 942, the communication means 940 and the link or path 941.

Still referring to FIG. 9, in one embodiment, the system controller 910 actuates or controls any of the micromechanical dispensing device 901 and the at least one additional dispensing device 902 based at least in part on the system sensor signal 935 that is formed by the system sensor 930.

Referring still to FIG. 9, in one embodiment the dispensing device 901 comprises the micromechanical dispensing device 600 described in connection with FIG. 6 above. This embodiment is now discussed in greater detail.

As described in connection with FIG. 6, the micromechanical dispensing device 600 is arranged to dispense a plurality of fluids 671, 672, 673 into the atmosphere. Accordingly, in this embodiment the dispensing system 900 is thus arranged to dispense the plurality of fluids 671, 672, 673 into the atmosphere.

With momentary cross-reference to FIG. 6, the dispensing device 901 comprises the micromechanical dispensing mechanism 610. As described in connection with FIG. 6, the micromechanical dispensing mechanism 610 is fluidly coupled to the included valve 665. The valve 665, in turn, is arranged to selectively fluidly couple the micromechanical dispensing mechanism 610 to a plurality of fluid reservoirs 620, 621, 622. The plurality of fluid reservoirs 620, 621, 622, in turn, comprise a corresponding plurality of fluids 671, 672, 673. As a result, the micromechanical dispensing device 600 is arranged to dispense the plurality of fluids 671, 672, 673 into the atmosphere.

In one embodiment, any of the plurality of fluid reservoirs 620, 621, 622 contain a fluid 671, 672, 673 comprising any of a fragrance, perfume, therapeutic, mood-enhancing agent, pheromone, moisturizer, humectant, miticide, deodorizer, disinfectant, sanitizing agent and insecticide.

As described in connection with FIG. 6, in one embodiment, the micromechanical dispensing mechanism 610 comprises any of an electrostatically-driven membrane, an electrostatically-actuated piston, a magnetically-actuated membrane, a thermally-actuated paddle vane and a ballistic aerosol dispensing mechanism.

As mentioned above, the dispensing system controller 910 actuates or controls the dispensing device 901 by means of suitable control signals that are communicated to the dispensing device 901 by means of the communication link or path 941, the communication means 940 and the communication link or path 943. These control signals, in turn, correspond to the program control signal 643 in FIG. 6. As described in connection with FIG. 6, the program control signal 643 is communicated to the device controller 640 comprised in the dispensing device 600 by means of the included controller interface 634 and the communication link or path 633.

In one embodiment, the dispensing device 901 further comprises the optional dispensing device sensor 660 in FIG. 6. As shown in the present FIG. 9, the dispensing device sensor 660 forms a system sensor signal 935′ (corresponding to the dispensing device sensor signal 635 as communicated to the dispensing device sensor interface 662 in FIG. 6) based on the atmospheric substance 980 and communicates the system sensor signal 935′ to the system controller 910 by means of the link or path 943, the communication means 940 and the link or path 941.

In one embodiment, the system controller 910 actuates or controls any of the micromechanical dispensing device 901 and the at least one additional dispensing device 902 based at least in part on the system sensor signal 935′ that is formed by the dispensing device sensor 660 of the dispensing device 901.

Referring still to FIG. 9, in one embodiment, the dispensing device 901 comprises the micromechanical dispensing device 700 described in connection with FIG. 7 above. This embodiment is now discussed in greater detail.

As described above in connection with FIG. 7, the micromechanical dispensing device 700 is arranged to dispense the fluid 771 into the atmosphere. Accordingly, in this embodiment, the dispensing system 900 depicted in the present FIG. 9 is thus arranged to dispense the fluid 771 into the atmosphere.

With momentary cross-reference to FIG. 7, the dispensing device 901 comprises the plurality of micromechanical dispensing mechanisms 710, 711, 712. As described in connection with FIG. 7, the plurality of micromechanical dispensing mechanisms 710, 711, 712 are fluidly coupled to the fluid reservoir 720. The fluid reservoir 720 comprises a corresponding fluid 771. As a result, the micromechanical dispensing device 700 is arranged to dispense the fluid 771 into the atmosphere.

In one embodiment, the fluid reservoir 720 contains a fluid 771 comprising any of a fragrance, perfume, therapeutic, mood-enhancing agent, pheromone, moisturizer, humectant, miticide, deodorizer, disinfectant, sanitizing agent and insecticide.

As described in connection with FIG. 7, in one embodiment, any of the plurality of micromechanical dispensing mechanisms 710, 711, 712 comprise any of an electrostatically-driven membrane, an electrostatically-actuated piston, a magnetically-actuated membrane, a thermally-actuated paddle vane and a ballistic aerosol dispensing mechanism.

As mentioned above, the dispensing system controller 910 actuates or controls the dispensing device 901 by means of suitable control signals that are communicated to the dispensing device by means of the communication link or path 941, the communication means 940 and the communication link or path 943. These control signals, in turn, correspond to the program control signal 743 in FIG. 7. As described in connection with FIG. 7, the program control signal 743 is communicated to the device controller 740 comprised in the dispensing device 700 by means of the included controller interface 734 and the communication link or path 733.

In one embodiment, the dispensing device 901 further comprises the optional dispensing device sensor 760 in FIG. 7. As shown in the present FIG. 9, the dispensing device sensor 760 forms a system sensor signal 935′ (corresponding to the dispensing device sensor signal 735 as communicated to the dispensing device sensor interface 762 in FIG. 7) based on the atmospheric substance 980 and communicates the system sensor signal 935′ to the system controller 910 by means of the link or path 943, the communication means 940 and the link or path 941.

In one embodiment, the system controller 910 actuates or controls any of the micromechanical dispensing device 901 and the at least one additional dispensing device 902 based at least in part on the system sensor signal 935′ that is formed by the dispensing device sensor 760 of the dispensing device 901.

Referring again generally to the present FIG. 9, the dispensing system 900 dispenses one or more fluids 671, 672, 673 when the dispensing device 901 comprises the micromechanical dispensing device 600. The dispensing system 900 dispenses the fluid 771 when the dispensing device 901 comprises the micromechanical dispensing device 700. Further, the dispensing system 900 dispenses one or more additional fluids 960 when the optional at least one additional dispensing device 902 is provided. Thus, in various embodiments the dispensing system 900 is capable of dispensing a wide variety of combinations and permutations of fluids into the atmosphere.

Referring now generally to the present FIG. 9, in one embodiment the atmospheric substance 980 comprises any of the one or more fluids that are dispensed by the dispensing system 900. Thus, when the dispensing device 901 comprises the micromechanical dispensing device 600 of FIG. 6, in one embodiment the atmospheric substance 980 comprises any of the plurality of fluids 671, 672, 673 that are dispensed by the micromechanical dispensing device 600 and the one or more fluids 960 that are dispensed by the optional at least one additional dispensing device 902. Further, when the dispensing device 901 comprises the micromechanical dispensing device 700 of FIG. 7, in one embodiment the atmospheric substance 980 comprises any of the fluid 771 that is dispensed by the micromechanical dispensing device 700 and the one or more fluids 960 that are dispensed by the optional at least one additional dispensing device 902.

Still referring generally to FIG. 9, in one embodiment the atmospheric substance 980 comprises any of a human body fluid in liquid or gaseous form and an odor or fragrance that is formed by a human body.

For example, the atmospheric substance 980 may comprise an odor or fragrance based on a state of mind (such as anxiety, fear or excitement) that is experienced by one or more humans. As another example, the odor or fragrance might comprise human perspiration or other human body odors.

Referring again generally to the present FIG. 9, in one embodiment the communication means 940 and the communication links or paths 941, 942, 943 and 944 comprise a communication network.

Still referring generally to FIG. 9, in one embodiment the communication means 940 and the links or paths 941, 942, 943 and 944 comprise any of a wireless network, an internet, a network hub, a telephone network, a local area network, a cable television network, a coaxial cable network, a fiber optics network, a satellite communication system, a universal serial bus, a universal serial bus port adapter and a twisted wire pair.

Referring still generally to FIG. 9, it is believed that all information, know-how and resources needed to enable the various communications between and amongst the components depicted therein and described above in connection therewith are common and well-known to those skilled in the art.

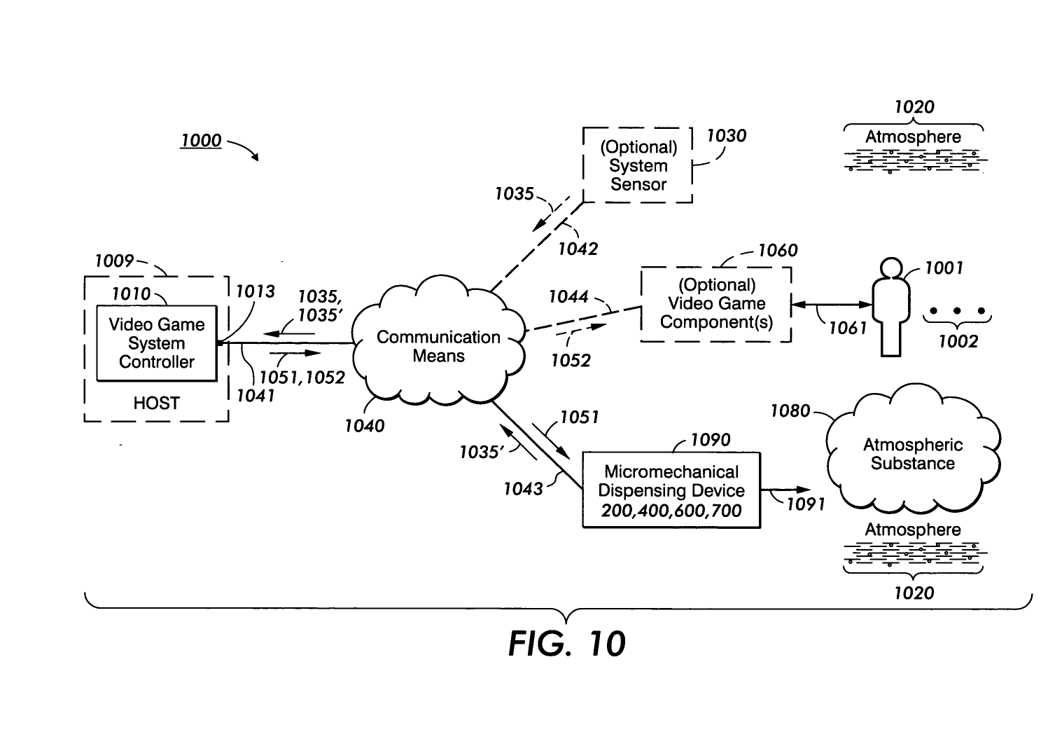

Referring now to FIG. 10, there is depicted a video game system 1000 including a video game system controller 1010 and a micromechanical dispensing device 1090. As shown, the micromechanical dispensing device 1090, in turn, comprises any of the micromechanical dispensing devices 200, 400, 600 and 700.

For good understanding, the micromechanical dispensing device 200 is described in connection with FIG. 2 above, the micromechanical dispensing device 400 is described in connection with FIG. 4 above, the micromechanical dispensing device 600 is described in connection with FIG. 6 above, and the micromechanical dispensing device 700 described in connection with FIG. 7 above.

The video game system controller 1010 is arranged to execute a video game program for the use of one or more video game players 1001, 1002.

The micromechanical dispensing device 1090 is arranged to dispense at least one fluid 1091 into an atmosphere 1020 under control of the video game system controller 1010.

In one embodiment, the one or more video game players 1001, 1002 are located in the atmosphere 1020.

As a result of the video game players 1001, 1002 being in the atmosphere 1020, the video game players 1001, 1002 influence the atmosphere 1020 and the atmosphere 1020 influences the video game players 1001, 1002. This is explained below.

First, the video game players 1001, 1002 influence the atmosphere 1020 as the video game players 1001, 1002 form various human body fluids in liquid and gaseous form including without limitation odors and fragrances. These human body fluids, in turn, are emitted into the atmosphere 1020 by various methods, including without limitation evaporation.

Second, the atmosphere 1020 influences the video game players 1001, 1002 as the video game players 1001, 1002 receive various substance that are comprised in the atmosphere 1020. These substances are received by various sensing methods, including detecting by human sensing organs such as, for example, the nose.

Further, as the video game system controller 1010 (under control of the video game program) causes the at least one fluid 1091 to be dispensed into the atmosphere 1020, then the video game system controller 1010 influences the video game players 1001, 1002.

As shown in FIG. 10, the video game system controller 1010 comprises a video game system controller communication interface 1013. Moreover, the video game system controller 1010 controls the micromechanical dispensing device 1090 by means of suitable control signals 1051 that are communicated to the micromechanical dispensing device 1090 by means of the communication interface 1013, a communication path or link 1041, an included communication means 1040 and a communication path or link 1043.

As shown, in one embodiment the video game system 1000 optionally comprises one or more video game components 1060 that are arranged to exchange video game information 1061 with the one or more video game players 1001, 1002. The video game system controller 1010 controls the one or more video game components 1060 by means of suitable control signals 1052 that are communicated to the video game components 1060 by means of the communication interface 1013, the path or link 1041, the communication means 1040 and a communication path or link 1044.

In one embodiment, the one or more video game components 1060 comprise one or more of any of the following: video display units, audio speakers, human hand control input devices, joysticks, keyboards, cursor control devices and computer mouse devices.

As shown in FIG. 10, in one embodiment the video game system controller 1010 is embodied in a video game system controller host 1009. In one embodiment, the video game system controller host 1009 comprises any of a video game console, a personal computer, a desktop computer, a laptop computer, a computing device, a communication device, a video game playing device, a personal digital assistant, a portable computing device, a portable communication device, a wireless phone, or the like.

With cross-reference to the Eliott patent, for example, in one embodiment the video game system controller host 1009 in the present FIG. 10 is similar or identical to the Eliott patent's video game console 52 comprising the main processor 100 that executes the video game program contained within the storage device 54 (game cartridge) as described in the Eliott patent from col. 9, line 19 to col. 15, line 60.

Referring again to FIG. 10, in one embodiment the video game program is provided to the video game system controller 1010 as embodied in a physical medium such as a game cartridge (as in the Eliott patent), a compact disk (CD), a DVD, or the like.

In one embodiment, the video game program is provided to the video game system controller 1010 remotely by means of electronic communication such as, for example, by being down-loaded from a remotely-located video game program source.

As explained in greater detail below, in various embodiments the video game system 1000 is arranged to form various system sensor signals 1035, 1035′, or both, based on a particular atmospheric substance 1080 that is comprised in the atmosphere 1020.

As shown in FIG. 10, in one embodiment the video game system 1000 comprises a system sensor 1030. For example, the system sensor 1030 may be similar or identical to the sensor of the Lewis patent.

The system sensor 1030 forms a system sensor signal 1035 based on the atmospheric substance 1080. The system sensor 1030 communicates the system sensor signal 1035 to the video game system controller 1010 by means of a communication path or link 1042, the communication means 1040 and the path or link 1041.

In one embodiment, the video game system controller 1010 controls the micromechanical dispensing device 1090 based on the system sensor signal 1035.

Also as shown in FIG. 10, in one embodiment the micromechanical dispensing device 1090 further comprises an integral dispensing device sensor 260, 460, 660 or 760. This is explained below.

Thus, in one embodiment the micromechanical dispensing device 1090 comprises the FIG. 2 micromechanical dispensing device 200. As described in connection with FIG. 2 above, the micromechanical dispensing device 200, in turn, includes the dispensing device sensor 260.

Further, in another embodiment the micromechanical dispensing device 1090 comprises the FIG. 4 micromechanical dispensing device 400. As described in connection with FIG. 4 above, the micromechanical dispensing device 400, in turn, includes the dispensing device sensor 460.

Also, in still another embodiment the micromechanical dispensing device 1090 comprises the FIG. 6 micromechanical dispensing device 600. As described in connection with FIG. 6 above, the micromechanical dispensing device 600, in turn, includes the dispensing device sensor 660.

Further, in yet another embodiment the micromechanical dispensing device 1090 comprises the FIG. 7 micromechanical dispensing device 700. As described in connection with FIG. 7 above, the micromechanical dispensing device 700, in turn, includes the dispensing device sensor 760.

As shown, in this latter embodiment the dispensing device sensor (260, 460, 660 or 760, as the case may be) comprised in the micromechanical dispensing device 1090 forms a system sensor signal 1035′ based on the atmospheric substance 1080. As shown, the dispensing device sensor comprised in the micromechanical dispensing device 1090 communicates the system sensor signal 1035′ to the video game system controller 1010 by means of the path or link 1043, the communication means 1040 and the path or link 1041.

In one embodiment, the video game system controller 1010 controls the micromechanical dispensing device 1090 based on the system sensor signal 1035′.

Referring generally to FIG. 10, in one embodiment the atmospheric substance 1080 comprises the at least one fluid 1091 that is dispensed by the micromechanical dispensing device 1090.

Still referring generally to FIG. 10, in one embodiment the atmospheric substance 1080 comprises a human body fluid in liquid or gaseous form.

Further, in one embodiment the atmospheric substance 1080 comprises an odor or fragrance that is formed by a human body.

Also, in one embodiment the atmospheric substance 1080 comprises a human body odor or fragrance that is formed by any of the one or more video game players 1001, 1002.

Referring to FIG. 10, as mentioned above, in a first embodiment the micromechanical dispensing device 1090 comprises the micromechanical dispensing device 200 described in connection with FIG. 2 above. Thus, with cross-reference to FIG. 2, in this first embodiment the micromechanical dispensing device 1090 comprises the one or more micromechanical dispensing mechanisms 210, 212 arranged to dispense one or more fluids 271, 273 into the atmosphere 1020, each of the one or more micromechanical dispensing mechanisms 210, 212 arranged to fluidly couple to a corresponding fluid reservoir of the one or more fluid reservoirs 220, 222.

In this first embodiment, any of the one or more fluid reservoirs 220, 222 contain a fluid 271, 273 comprising any of a fragrance, perfume, therapeutic, mood-enhancing agent, pheromone, moisturizer and humectant.

In this first embodiment, any of the one or more micromechanical dispensing mechanisms 210, 212 comprise any of an electrostatically-driven membrane, an electrostatically-actuated piston, a magnetically-actuated membrane, a thermally-actuated paddle vane and a ballistic aerosol dispensing mechanism.

Still referring to FIG. 10, as mentioned above, in a second embodiment the micromechanical dispensing device 1090 comprises the micromechanical dispensing device 400 described in connection with FIG. 4 above. Thus, with cross-reference to FIG. 4, in this second embodiment the micromechanical dispensing device 1090 comprises the plurality of micromechanical dispensing mechanisms 410, 411, 412 arranged to dispense a plurality of fluids 471, 472, 473 into the atmosphere 1020, each of the plurality of micromechanical dispensing mechanisms arranged to fluidly couple to a corresponding fluid reservoir of the plurality of fluid reservoirs 420, 421, 422.

In this second embodiment, any of the plurality of fluid reservoirs 420, 421, 422 contain a fluid 471, 472, 473 comprising any of a fragrance, perfume, therapeutic, mood-enhancing agent, pheromone, moisturizer and humectant.

In this second embodiment, any of the plurality of micromechanical dispensing mechanisms 410, 411, 412 comprise any of an electrostatically-driven membrane, an electrostatically-actuated piston, a magnetically-actuated membrane, a thermally-actuated paddle vane and a ballistic aerosol dispensing mechanism.

Still referring to FIG. 10, as mentioned above, in a third embodiment the micromechanical dispensing device 1090 comprises the micromechanical dispensing device 600 described in connection with FIG. 6 above. Thus, with cross-reference to FIG. 6, in this third embodiment the micromechanical dispensing device 1090 comprises the micromechanical dispensing mechanism 610 arranged to dispense a plurality of fluids 671, 672, 673 into the atmosphere 1020, the micromechanical dispensing mechanism being fluidly coupled to the included valve 665, wherein the valve is arranged to selectively fluidly couple the micromechanical dispensing mechanism 610 to the plurality of fluid reservoirs 620, 621, 622.

In this third embodiment, any of the plurality of fluid reservoirs 620, 621, 622 contain a fluid 671, 672, 673 comprising any of a fragrance, perfume, therapeutic, mood-enhancing agent, pheromone, moisturizer and humectant.

In this third embodiment, the micromechanical dispensing mechanism 610 comprises any of an electrostatically-driven membrane, an electrostatically-actuated piston, a magnetically-actuated membrane, a thermally-actuated paddle vane and a ballistic aerosol dispensing mechanism.

Still referring to FIG. 10, as mentioned above, in a fourth embodiment the micromechanical dispensing device 1090 comprises the micromechanical dispensing device 700 described in connection with FIG. 7 above. Thus, with cross-reference to FIG. 7, in this fourth embodiment the micromechanical dispensing device 1090 comprises the plurality of micromechanical dispensing mechanisms 710, 711, 712 arranged to dispense a fluid 771 into the atmosphere 1020, the plurality of micromechanical dispensing mechanisms arranged to fluidly couple to the fluid reservoir 720.

In this fourth embodiment, the fluid reservoir 720 contains a fluid 771 comprising any of a fragrance, perfume, therapeutic, mood-enhancing agent, pheromone, moisturizer and humectant.

In this fourth embodiment, any of the plurality of micromechanical dispensing mechanisms 710, 711, 712 comprise any of an electrostatically-driven membrane, an electrostatically-actuated piston, a magnetically-actuated membrane, a thermally-actuated paddle vane and a ballistic aerosol dispensing mechanism.

Referring now generally to the present FIG. 10, in one embodiment the communication means 1040 and the path or links 1041, 1042, 1043 and 1044 comprise any of a local area network, local channels, local wiring, local cabling, wide area network, wireless network, internet, network hub, telephone network, cable television network, coaxial cable network, fiber optics network, satellite communication system, universal serial bus, universal serial bus port adapter and twisted wire pair.

Still referring generally to the present FIG. 10, it is believed that all information, know-how and resources needed to enable the various communications between and amongst the components depicted therein and described above in connection therewith are common and well-known to those skilled in the art.

Referring still generally to the present FIG. 10, various applications of the video game system 1000 are now discussed.

In one application, the video game system controller 1010 (under control of the video game program) causes the micromechanical dispensing device 1090 to dispense odors or fragrances to enhance the video game players 1001, 1002's enjoyment of the video game.

In another application, the video game system controller 1010 causes the micromechanical dispensing device 1090 to dispense odors or fragrances to enhance or modify the video game players 1001, 1002's reaction to the action or events depicted in the video game.

In a further application, the video game system controller 1010 causes the micromechanical dispensing device 1090 to dispense odors or fragrances related to the background setting of the action or events depicted in the video game.

For example, if an auto racing video game uses a racetrack background setting, then racetrack fragrances are dispensed. As a variant, if the auto racing video game is depicted in a city streets background setting, then city street fragrances are dispensed.

As another example, if a sporting event video game uses an outdoor stadium background setting, then outdoor stadium fragrances are dispensed. As a variant, if the sporting event video game uses an indoor stadium background setting, then indoor stadium fragrances are dispensed.

In still another application, the video game system controller 1010 causes the micromechanical dispensing device 1090 to dispense odors or fragrances that are associated with the action or events depicted in the video game. For example, certain actions or events have characteristic odors or fragrances.

In a still further application, the video game system controller 1010 (under control of the video game program) is arranged to detect a situation or event arising from the video game players 1001, 1002 use of the video game system.

Thus, in one example, the video game system controller 1010 is programmed to detect (by means of any of the sensing devices described herein) the odor, fragrance or smell of fear, anxiety or tension that is emitted by the video game players 1001, 1002 and to react by causing the micromechanical dispensing device 1090 to dispense a calming odor or fragrance to counter-act the fear, anxiety or tension in the players. In a variant, the dispensing device 1090 dispenses pheromones to control the situation.

In another application, the video game system controller 1010 (under control of the video of the video game program) generates a desired mood or emotional state in the video game players 1001, 1002 by causing the micromechanical dispensing device 1090 to dispense a mood-enhancing agent. In a variant, the controller 1010 detects (by means of an associated sensing device) when the desired mood or emotional state in the video game players 1001, 1002 is achieved.

Referring now generally to the embodiments described in connection with FIGS. 2-10 above, in one application any of these embodiments are used to dispense fragrances.

The domestic home fragrance market in 2000 was 2.2 billion dollars. This market is high-value, emotive-driven, and demands the latest high tech, trendy and “hip” delivery devices. Various technologies have been used to deliver fragrances including candles, diffusers, room sprays and ultrasonic nebulizers. Candles and diffusers typically require heating of the fragrance in order to disperse the fragrance, and are not amenable to digital delivery-on-demand as might be required in applications such as a digital “smell synthesizer” or a closed-loop olfactory system that includes an electronic smell detector or “nose” and a digital smell actuator. While existing thermal fluid ejectors could be used to provide a drop-on-demand fragrancer, it would also heat the fragrance which, in turn, could cause chemical changes in the odor. Ultrasonic nebulizers and sprayers work without heating the fragrance, but are not amenable to drop-on-demand with well-controlled doses. These latter devices are also not easily integrated with control and feedback electronics as might be required for a smell synthesizer.

In addition, other potential applications for the embodiments of FIGS. 2-10 include dispensing any of perfumes, therapeutics, mood-enhancing agents, pheromones, moisturizers, humectants, miticides, deodorizers, disinfectants, sanitizing agents and insecticides.

One key advantage of the embodiments of FIGS. 2-10 is that the included micromechanical dispensing devices are able to deliver multiple fragrances on demand without the need for heating the fragrance. Since the micromechanical dispensing devices can be used to control the dosage by means of electronic control signals, they can be used in systems such as a digital smell synthesizer or closed loop olfactory system. In addition, the micromechanical dispensing devices can be fabricated using microelectronic batch fabrication in order to decrease the cost of the fluid actuator, thus opening new fragrance-dispensing markets.

In one application, any of the embodiments of FIGS. 2-10 are arranged to sense and react to various situations. Thus, a fragrance dispenser, such as any of the micromechanical dispensing devices described herein, is placed at a particular location in a room, and an electronic nose, such as any of the sensing devices described herein, is placed at a different location where the smell is to be controlled to determine the concentration of the fragrance at this remote location. An included controller actuates the fragrance dispenser until a set-point fragrance concentration is reached at the remote location.

In one application, an electronic nose, such as any of the sensing devices described herein, is arranged to detect the presence of an undesirable or foul odor and to react by causing an associated fluid dispenser, such as any of the micromechanical dispensing devices described herein, to dispense a pleasant odor. In a variant, this arrangement is used in refrigerators to counteract the unpleasant odors that are currently treated with an open box of baking soda. In another variant, this arrangement is used in toilets and washrooms to address unpleasant situations.

In one application, an electronic nose, such as any of the sensing devices described herein, is arranged to detect a situation arising from a human meeting, such as the smell of fear, anxiety or tension, and to react by causing an associated fluid dispenser, such as any of the micromechanical dispensing devices described herein, to dispense a calming odor to counter-act the fear, anxiety or tension. In a variant, the fluid dispenser dispenses pheromones to control the situation.

In summary, there has been described the first aspect of the invention, namely, an environmental system 800A including a micromechanical dispensing device 801, the environmental system 800A comprising an environmental system controller 810 arranged to communicate with one or more included environmental air units 860, 861, the one or more environmental air units 860, 861 arranged to alter or control one or more physical properties of an atmosphere 820, the environmental system controller 810 further arranged to communicate with an included micromechanical dispensing device 801, the micromechanical dispensing device 801 arranged to dispense one or more fluids 271, 273 into the atmosphere 820, the micromechanical dispensing device 801 comprising one or more micromechanical dispensing mechanisms 210, 212, each of the one or more micromechanical dispensing mechanisms 210, 212 arranged to fluidly couple 254, 255 to a corresponding fluid reservoir of one or more fluid reservoirs 220, 222.

Also, there has been described the second aspect of the invention, namely, an environmental system 800B including a micromechanical dispensing device 802, the environmental system 800B comprising an environmental system controller 810 arranged to communicate with one or more included environmental air units 860, 861, the one or more environmental air units 860, 861 arranged to alter or control one or more physical properties of an atmosphere 820, the environmental system controller 810 further arranged to communicate with an included micromechanical dispensing device 802, the micromechanical dispensing device 802 arranged to dispense a plurality of fluids 471, 472, 473 into the atmosphere 820, the micromechanical dispensing device 802 comprising a plurality of micromechanical dispensing mechanisms 410, 411, 412, each of the plurality of micromechanical dispensing mechanisms 410, 411, 412 arranged to fluidly couple 454, 455, 456 to a corresponding fluid reservoir of a plurality of fluid reservoirs 420, 421, 422.

Also, there has been described the third aspect of the invention, namely, an environmental system 800C including a micromechanical dispensing device 803, the environmental system 800C comprising an environmental system controller 810 arranged to communicate with one or more included environmental air units 860, 861, the one or more environmental air units 860, 861 arranged to alter or control one or more physical properties of an atmosphere 820, the environmental system controller 810 further arranged to communicate with an included micromechanical dispensing device 803, the micromechanical dispensing device 803 arranged to dispense a plurality of fluids 671, 672, 673 into the atmosphere 820, the micromechanical dispensing device 803 comprising a micromechanical dispensing mechanism 610 that is fluidly coupled to an included valve 665, wherein the valve 665 is arranged to selectively fluidly couple 611-611′, 612 the micromechanical dispensing mechanism 610 to a plurality of fluid reservoirs 620, 621, 622.

Also, there has been described the fourth aspect of the invention, namely, an environmental system 800D including a micromechanical dispensing device 804, the environmental system 800D comprising an environmental system controller 810 arranged to communicate with one or more included environmental air units 860, 861, the one or more environmental air units 860, 861 arranged to alter or control one or more physical properties of an atmosphere 820, the environmental system controller 810 further arranged to communicate with an included micromechanical dispensing device 804, the micromechanical dispensing device 804 arranged to dispense a fluid 771 into the atmosphere 820, the micromechanical dispensing device 804 comprising a plurality of micromechanical dispensing mechanisms 710, 711, 712 arranged to fluidly couple 754 to a fluid reservoir 720.

Also, there has been described the fifth aspect of the invention, namely, an environmental system (comprising any of the environmental systems 800A, 800B, 800C and 800D) including a micromechanical dispensing device (comprising any of the dispensing devices 801, 802, 803 and 804), the environmental system comprising an environmental system controller 810 arranged to communicate with one or more included environmental air units 860, 861, the one or more environmental air units 860, 861 arranged to alter or control one or more physical properties of an atmosphere 820, the environmental system controller 810 further arranged to communicate with an included micromechanical dispensing device (that is, any of the dispensing devices 801, 802, 803 and 804), the micromechanical dispensing device arranged to dispense at least one fluid (that is, any of the fluids 271, 273, 471, 472, 473, 671, 672, 673, 771) into the atmosphere 820.

While various embodiments of an environmental system including a micromechanical dispensing device, in accordance with the present invention, have been described above, the scope of the invention is defined by the claims below.

What is claimed is:

1. An environmental system including a micromechanical dispensing device, the environmental system comprising:
an environmental system controller arranged to communicate with one or more included environmental air units, the one or more environmental air units arranged to alter or control one or more physical properties of an atmosphere,
the environmental system controller further arranged to communicate with an included micromechanical dispensing device, the micromechanical dispensing device arranged to dispense a plurality of fluids into the atmosphere,
the micromechanical dispensing device comprising a micromechanical dispensing mechanism that is fluidly coupled to an included valve, wherein the valve is arranged to selectively fluidly couple the micromechanical dispensing mechanism to a plurality of fluid reservoirs.

2. The environmental system of claim 1, wherein any of the plurality of fluid reservoirs contain a fluid comprising any of a fragrance, perfume, therapeutic, mood-enhancing agent, pheromone, moisturizer, humectant, miticide, deodorizer, disinfectant, sanitizing agent and insecticide.

3. The environmental system of claim 1, wherein the micromechanical dispensing mechanism comprises any of an electrostatically-driven membrane, an electrostatically-actuated piston, a magnetically-actuated membrane, a thermally-actuated paddle vane and a ballistic aerosol dispensing mechanism.

4. The environmental system of claim 1 further comprising a system sensor arranged to form a system sensor signal based on an atmospheric substance comprised in the atmosphere and to communicate the system sensor signal to the environmental system controller.

5. The environmental system of claim 4, wherein the environmental system controller is arranged to control any of the micromechanical dispensing device and the one or more environmental air units based on the system sensor signal.

6. The environmental system of claim 5, wherein the atmospheric substance comprises any of a human body fluid in liquid or gaseous form, an odor or fragrance that is formed by a human body and the plurality of fluids that are dispensed by the micromechanical dispensing device.

7. The environmental system of claim 1, wherein the micromechanical dispensing device further comprises a dispensing device sensor arranged to form a system sensor signal based on an atmospheric substance comprised in the atmosphere and to communicate the system sensor signal to the environmental system controller.

8. The environmental system of claim 7, wherein the environmental system controller is arranged to control any of the micromechanical dispensing device and the one or more environmental air units based on the system sensor signal.

9. The environmental system of claim 8, wherein the atmospheric substance comprises any of a human body fluid in liquid or gaseous form, an odor or fragrance that is formed by a human body and the plurality of fluids that are dispensed by the micromechanical dispensing device.

10. The environmental system of claim 1, wherein any of the one or more environmental air units are arranged to alter or control any of the following included physical properties of the atmosphere: temperature, humidity, circulation and cleanliness.
